(19) United States
(12) Patent Application Publication
(10) Pub. No.: US 2023/0084937 A1
(43) Pub. Date: Mar. 16, 2023

(54) METHODS FOR TREATING PATIENTS HAVING SINUSOIDAL OBSTRUCTION SYNDROME

(71) Applicant: Gentium S.R.L., VILLA GUARDIA (CO) (IT)

(72) Inventor: Massimo IACOBELLI, Milano (IT)

(21) Appl. No.: 17/883,452

(22) Filed: Aug. 8, 2022

Related U.S. Application Data
(63) Continuation of application No. 16/398,978, filed on Apr. 30, 2019, now abandoned.
(60) Provisional application No. 62/664,657, filed on Apr. 30, 2018.

(51) Int. Cl. A61K 31/711; A61P 1/16; A61K 9/00

(52) U.S. Cl. CPC A61K 31/711; A61P 1/16; A61K 9/0019

(57) ABSTRACT

In various embodiments, the present disclosure teaches methods of treating patients diagnosed as having sinusoidal obstruction syndrome with defibrotide.

### CROSS REFERENCE TO RELATED APPLICATIONS

This application is a continuation of U.S. Utility Pptent application Ser. No. 16/398,978, filed Apr. 30, 2019, which claims the benefit of U.S. Provisional Application No. 62/664,657, filed on Apr. 30, 2018, the contents of which is hereby incorporated by reference in their entirety.

### FIELD OF THE INVENTION

Disclosed embodiments relate generally to methods for treating a patient diagnosed as having sinusoidal obstruction syndrome. More particularly, disclosed embodiments relate to methods for treating a patient diagnosed as having late-onset sinusoidal obstruction syndrome, comprising administering a therapeutically effective amount of defibrotide to the patient. Other embodiments relate to a method for treating a patient diagnosed as having sinusoidal obstruction syndrome, comprising administering a therapeutically effective amount of defibrotide to the patient, where the patient has undergone at least one chemotherapy treatment and has not undergone hematopoietic stem cell transplantation. Further embodiments relate to prophylactic defibrotide administration to patients at risk of developing sinusoidal obstruction syndrome.

### BACKGROUND

Hematopoietic stem cell transplantation (HSCT) is a potentially life-saving treatment for many patients with inherited disorders and hematologic malignancies. However, its practical use is limited by the risk of serious adverse events, including sinusoidal obstruction syndrome (SOS, the now preferred name for veno-occlusive disease (VOD)). SOS typically occurs as a result of HSCT, however it has been observed to occur after primary chemotherapy as well. While the incidence of SOS after primary chemotherapy is not well documented, it has been reported before, for example in 11% of patients receiving inotuzumab with and without HSCT. (Kantarjian et al. 2016).

SOS has a reported mean incidence of 13.7% post-HSCT. (Coppell et al. 2010) Typical symptoms of SOS may include hyperbilirubinemia, painful hepatomegaly, weight gain, and ascites. SOS is a life-threatening liver injury complication with greater than 80% mortality in severe cases that affects up to 20% of allogeneic HSCT recipients in some centers. Severe SOS is historically defined by concurrent multi-organ dysfunction (MOD). SOS can also occur after intense chemotherapy when either the chemotherapy or radiation induces both systemic inflammation and tissue damage particularly to the sinusoidal endothelial cells of the hepatic acinus. In addition, SOS can also occur after use of drugs such as inotuzumab, 6-thioguanine, actinomycin-D, gemtuzumab, ozogamicin, and the combination of tacrolimus and sirolimus. (Wadleigh et al. 2003).

SOS may progress along a pathophysiologic cascade that involves injury to hepatocytes, endothelial cells, and stellate cells; augmented fibrogenesis, excessive fibrin deposition, and ischemia; injury to pulmonary or renal tissues, and multi-organ dysfunction (MOD; typically, respiratory and/or renal failure). (Mohty et al. 2015).

Defibrotide is currently the only therapy approved in the United States for the treatment of adult and pediatric patients with hepatic SOS with renal or pulmonary dysfunction following HSCT and in the European Union for treatment of severe SOS post-HSCT in adults and in children (aged >1 month). (Defitelio Summary of Product Characteristics, 2016). In preclinical and human studies, defibrotide has demonstrated profibrinolytic, antithrombotic, anti-inflammatory, and pro-angiogenic actions, thus promoting an anticoagulant phenotype of the endothelium that protects and stabilizes the function of endothelial cells. (Mohty et al. 2015).

SOS has historically been diagnosed by the Baltimore or modified Seattle criteria. (Jones et al. 1987). The Baltimore criteria require bilirubin increases to ≥2 mg/dL (≥34 μmol/L) within 21 days of HSCT with 2 or more of the following symptoms: ascites, painful hepatomegaly, and weight gain (≥5% baseline weight). The modified Seattle criteria require 2 or more of the following within 20 days of HSCT: bilirubin increases to >2 mg/dL (≥34 μmol/L), ascites, increased hepatomegaly over baseline, and weight gain of >2% above baseline. (Corbacioglu et al. 2012).

Late-onset SOS has been reported beyond the 20-21 days after HSCT specified in the original Baltimore or modified Seattle diagnostic criteria, and may occur in 15%-20% of cases. (O'Donnell et al. 1999). Late-onset SOS has been associated with poorer outcome in some reports (SOS>30 days after HSCT) but not in others (SOS>20 days after HSCT). (Hasegawa et al. 2012). Diagnosis of late-onset SOS also may occur after hospital discharge, potentially delaying treatment. (Carreras et al. 2007).

Furthermore, for a variety of reasons, patients having SOS oftentimes need to delay defibrotide treatment.

Thus, a need exists for treating patients having late-onset SOS, which goes undetected by current diagnostic methods or for treating patients who delayed defibrotide treatment upon SOS diagnosis following primary chemotherapy.

Additionally, better than treating SOS after it develops, methods of preventing its development in the first place are being studied. Currently, there are no drugs approved for prophylaxis of SOS, and evidence for most therapies is limited (Cheuk et al. 2015). Use of heparin prophylaxis is controversial: a 2006 meta-analysis of 12 studies found no statistically significant reduction in SOS risk (Imran et al. 2006). However, two of three randomized controlled trials included in the meta-analysis reported a significant decrease in the incidence of SOS. (Mohty et al. 2015; Attal et al. 1992; Or et al. 1996; Marsa-Vila et al. 1991). Data on prophylaxis with ursodeoxycholic acid are inconclusive; some studies found a significant benefit, and others have not found a benefit in reducing the incidence of SOS. (Mohty et al. 2015; Dingan et al. 2014; Ruutu et al. 2002; Tay et al. 2007). Of note, ursodeoxycholic acid reduces bilirubin levels via stimulation of bile acid secretion, but this change in bilirubin may not be therapeutic. (Tsochatzis et al. 2013). Finally, a pediatric phase 3 randomized controlled trial (RCT) of prophylaxis in high-risk pediatric patients found reduced incidence of SOS in patients undergoing HSCT receiving prophylactic defibrotide. (Corbacioglu et al. 2012). Thus, a need exists for prophylactic treatment to prevent or otherwise the development of SOS.

### SUMMARY

Methods for treating a patient diagnosed as having sinusoidal obstruction syndrome are described herein. In some embodiments, the present disclosure provides methods of treating a patient diagnosed as having sinusoidal obstruction syndrome, comprising administering a therapeutically effective amount of defibrotide to the patient, where the patient is from about 0 years of age to about 16 years of age, and where the patient was diagnosed as having sinusoidal obstruction syndrome at least 21 days after the patient has undergone hematopoietic stem cell transplantation, and where one or more administrations of the defibrotide are given to the patient over a treatment period.

### BRIEF DESCRIPTION OF THE DRAWINGS

FIG. 1 is a graph showing the Kaplan-Meier Day +100 survival for patients in the SOS with MOD population, SOS without MOD population, and the overall HSCT population.

FIG. 2A is a graph showing the Kaplan-Meier Day +100 survival for pediatric patients in the SOS with MOD population, SOS without MOD population and the overall SOS population. FIG. 2B is a graph showing the Kaplan-Meier Day +100 survival for pediatric patients in the SOS with MOD population, SOS without MOD population and the overall SOS population. FIG. 2C is a graph showing the Kaplan-Meier Estimated Day +100 survival rates by age group.

FIGS. 3A-3C are graphs showing the Kaplan-Meier Estimated Day +100 Survival Rates for all late-onset SOS patients (A), late-onset patients with MOD (B), and late-onset patients without MOD (C). FIGS. 3D and 3E are graphs showing the Kaplan-Meier Estimated Day +100 Survival Rates for the overall pediatric population (D) and the late-onset pediatric population (E).

FIG. 4. is a graph showing the Kaplan-Meier Estimated Day +100 survival rates by age group for patients with normal-onset SOS and late-onset SOS.

FIG. 5 is a graph showing the timing of initiation of defibrotide by day post-diagnosis.

FIG. 6 is a graph showing the survival rates at Day +100 by day of initiation of defibrotide.

FIGS. 7A and 7B are graphs showing the survival rates at Day +100 survival trends across initiation day for all patients (A) and patients with MOD (B).

FIGS. 8A and 8B are graphs showing Kaplan-Meier Estimated Survival for patients with and without MOD (A) and pediatric and adult patients (B).

### DETAILED DESCRIPTION

Methods for treating a patient diagnosed as having sinusoidal obstruction syndrome are described herein. In some embodiments, the present disclosure provides methods of treating a patient diagnosed as having sinusoidal obstruction syndrome, comprising administering a therapeutically effective amount of defibrotide to the patient, where the patient is from about 0 years of age to about 16 years of age, and where the patient was diagnosed as having sinusoidal obstruction syndrome at least 21 days after the patient has undergone hematopoietic stem cell transplantation, and where one or more administrations of the defibrotide are given to the patient over a treatment period. In some embodiments, the present disclosure provides methods of treating a patient diagnosed as having sinusoidal obstruction syndrome, comprising administering a therapeutically effective amount of defibrotide to the patient, where the patient has undergone at least one chemotherapy treatment and has not undergone hematopoietic stem cell transplantation, and where one or more administrations of the defibrotide are given to the patient over a treatment period.

In some embodiments, the present disclosure provides methods of prophylactic treatment of a patient receiving hematopoietic stem cell transplantation, comprising administering a therapeutically effective amount of defibrotide to the patient, and where one or more administrations of the defibrotide are given to the patient over a treatment period. In some embodiments, the patient is at high-risk or very high-risk of developing sinusoidal obstruction syndrome. In some embodiments, the defibrotide is administered before conditioning for hematopoietic stem cell transplantation and/or HSCT begins. In some embodiments, the defibrotide is administered with conditioning for hematopoietic stem cell transplantation and/or HSCT. In some embodiments, the defibrotide is administered after conditioning for hematopoietic stem cell transplantation has begun, but before development of symptoms related to SOS. In some embodiments, the defibrotide is administered one day before conditioning for hematopoietic stem cell transplantation begins.

In some embodiments, “high-risk” patients meet both (i and ii) of the following criteria: i) patient must be scheduled to receive myeloablative conditioning, and ii) meet one of the following criteria: a) have at least one hepatic related risk factor; or b) have advanced stage-neuroblastoma requiring myeloablative conditioning. In some embodiments, the high-risk patient is an adult patient. In some embodiments, the adult patient is older than 16 years old.

Hepatic risk factors may be defined by the European Society for Blood and Marrow Transplantation (EBMT) position statement during screenings as follows: 1) transaminase level >2.5 times the upper limit of normal (ULN) during screening or within 14 days prior to screening on a non-screening test if the test was performed as part of the patient's routine standard of care; 2) serum total bilirubin level >1.5 times the ULN during screening or within 14 days prior to screening on a non-screening test if the test was performed as part of the patient's routine standard of care; 3) prior history of cirrhosis (with biopsy evidence); 4) prior history of hepatic fibrosis (by histology or other diagnostic scoring system); 5) prior history of viral hepatitis within one year before the start of the study treatment, as indicated by an available positive test result for any of the following: hepatitis A virus (HAS) immunoglobulin M (IgM) (anti-HAV IgM), hepatitis B virus (HBV) core immunoglobulin G (IgG) or IgM (anti-HBc IgG or anti-HBc IgM), HBV surface antigen (HBsAg), HBV deoxyribonucleic acid (DNA) by polymerase chain reaction or nucleic acid amplification testing (NAAT), hepatitis C virus (HCV) antibody (anti-HCV) and HCV RNA by PCR or NAAT; 6) any prior hepatic irradiation, including abdominal irradiation covering the hepatic area; or 7) documented diagnosis of iron overload (serum ferritin >2000 ng/mL or liver iron content ≥5.0 mg/gdw as estimated by magnetic resonance imaging T2* within three months prior to screening.

Myeloablative conditioning may be defined as 1) at least two alkylating agents (e.g. cyclophosphamide, busulfan, melphalan) or 2) total body irradiation (TBI) (e.g. a single dose of ≥5Gy, or ≥8 Gy fractionated dose) and at least one alkylating agent.

In some embodiments, “very high-risk” patients must meet one of the following criteria: 1) osteopetrosis and undergoing myeloablative conditioning; 2) primary hemophagocytic lymphohistiocytosis (HLH), Griscelli II Chediak-Higashi syndrome, Hermansky-Pudiak II, X-linked lymphoproliferative disorders, X-linked severe combined immunodeficiency, X-linked hypogammaglobulinemia, or familial HLH 1-5 and undergoing myeloablative conditioning; 3) prior treatment with an ozogamicin-containing monoclonal antibody using the minimum dose and schedule (e.g. Gemtuzumab ozogamicin, at least 9 mg/m2 total dose or Inotuzumab ozogamicin, at least 1.5 mg/mg2 over 28 days); or 4) class III, high-risk thalassemia (i.e. patients who are over seven years old and have a liver size ≥5 cm at the time of screening). In some embodiments, the very high-risk patient is a pediatric patient.

In some embodiments, the present disclosure provides methods of treatment of a patient receiving chemotherapy, comprising administering a therapeutically effective amount of defibrotide to the patient, and where one or more administrations of the defibrotide are given to the patient over a treatment period. In some embodiments, the defibrotide is administered after the chemotherapy patient develops symptoms of SOS or is diagnosed with SOS. In some embodiments, the defibrotide is administered before the chemotherapy patient develops symptoms of SOS or is diagnosed with SOS. In some embodiments, the defibrotide is administered at the initiation of chemotherapy. In some embodiments, the defibrotide is administered before the initiation of chemotherapy. In some embodiments, the chemotherapy is a immunoablative chemotherapy.

Definitions

The term defibrotide identifies a polydeoxyribonucleotide that is obtained by extraction from animal and/or vegetable tissues but which may also be produced synthetically; the polydeoxyribonucleotide is normally used in the form of an alkali-metal salt, generally a sodium salt, and generally has a molecular weight of 13 to 30 kDa (CAS Registry Number: 83712-60-1). Preferably, defibrotide is obtained according to U.S. Pat. Nos. 4,985,552 and 5,223,609 and/or presents the physical/chemical characteristics described in the same U.S. Pat. Nos. 4,985,552 and 5,223,609, herein incorporated by reference. More in particular, defibrotide is a mixture of polydeoxyribonucleotides having formula of random sequence: P1-5, (dAP)₁₂₋₂₄, (dGP)₁₀₋₂₀, (dPp)₁₃₋₂₆, (dCP)₁₀₋₂₀, where: P=phosphoric radical; dAp=deoxyadenylic monomer; dGp=deoxyguanylic monomer; dTp=deoxythymidinic monomer; dCp=deoxycytidynic monomer; and/or shows the following chemical/physical characteristics: electrophoresis=homogeneous anodic mobility, and/or extinction coefficient, E₁ cm^{1%} at 260±1 nm nm=220±10, and/or E₂₃₀/E₂₆₀=0.45±0.04, and/or coefficient of molar extinction (referred to phosphorous) ε(P)=7.750±500, and/or rotatory power [α]_{D}^{20°}=53°±6; and/or reversible hyperchromicity, indicated as % in native DNA and/or h=15±5.

The term “subject” is used interchangeably herein with “patient” to refer to an individual to be treated. The subject is a mammal (e.g., human, non-human primate, rat, mouse, cow, horse, pig, sheep, goat, dog, cat, etc.). The subject can be a clinical patient, a clinical trial volunteer, an experimental animal, etc. The subject can be suspected of having or at risk for having a condition (such as SOS) or be diagnosed with a condition (such as SOS). The subject can also be suspected of having or at risk for having SOS. According to one embodiment, the subject to be treated according to this invention is a human. Subjects to be treated by the methods of the disclosed embodiments include both human subjects and animal subjects (e.g., dog, cat, monkey, chimpanzee, and/or the like) for veterinary purposes. The subjects may be male or female and may be any suitable age, e.g., neonatal, infant, juvenile, adolescent, adult, or geriatric. In some embodiments, the subjects are preferably mammalian.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

A “therapeutically effective amount”, as used herein refers to an amount that is sufficient to achieve a desired therapeutic effect. For example, a therapeutically effective amount can refer to an amount that is sufficient to improve at least one sign or symptom of SOS.

The terms “a” and “an,” when used to modify the ingredient of a composition, such as, active agent, buffering agent, and osmolyte, do not denote a limitation of quantity, but rather denote the presence of at least one of the referenced item. The term “or” or “and/or” is used as a function word to indicate that two words or expressions are to be taken together or individually. The terms “comprising,” “having,” “including,” and “containing” are to be construed as open-ended terms (i.e., meaning “including, but not limited to”). The endpoints of all ranges directed to the same component or property are inclusive and independently combinable.

Throughout the present specification, the terms “about” and/or “approximately” may be used in conjunction with numerical values and/or ranges. The term “about” is understood to mean those values near to a recited value. For example, “about 1200 [units]” may mean within ±10% of 1200, within ±10%, ±9%, ±8%, ±7%, ±7%, ±5%, ±4%, ±3%, ±2%, ±1%, less than ±1%, or any other value or range of values therein. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 70-70, etc.). Furthermore, all values within a given range may be an endpoint for the range encompassed thereby (e.g., the range 50-80 includes the ranges with endpoints such as 55-80, 50-75, etc.).

In some embodiments, the present disclosure provides a method for treating a patient diagnosed as having sinusoidal obstruction syndrome, comprising administering a therapeutically effective amount of defibrotide to the patient, where the patient is from about 0 years of age to about 16 years of age, and where the patient was diagnosed as having sinusoidal obstruction syndrome at least 21 days after the patient has undergone hematopoietic stem cell transplantation, and where one or more administrations of the defibrotide are given to the patient over a treatment period. Accordingly, in some embodiments, the patient was not diagnosed as having sinusoidal obstruction syndrome within 21 days after the patient has undergone hematopoietic stem cell transplantation. The patient diagnosed as having sinusoidal obstruction syndrome may or may not have multiple organ dysfunction. Thus, in some embodiments, the patient has multiple organ dysfunction. In some embodiments, the patient does not have multiple organ dysfunction.

In certain embodiments, the one or more administrations of the defibrotide begins on or after 22 days after the patient has undergone hematopoietic stem cell transplantation. Thus, in some embodiments, the administrations of the defibrotide begins on or after 22, 23, 24, 25, 26, 27, 28, or 29 days after the patient has undergone hematopoietic stem cell transplantation. In certain embodiments, the one or more administrations of the defibrotide begins on or after 30 days after the patient has undergone hematopoietic stem cell transplantation. Thus, in some embodiments, the administration of the defibrotide begins on or after 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days after the patient has undergone hematopoietic stem cell transplantation.

In certain embodiments, the one or more administrations of the defibrotide begins on or after 30 days after the patient has undergone hematopoietic stem cell transplantation. Thus, in some embodiments, the administration of the defibrotide begins on or after 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days after the patient has undergone hematopoietic stem cell transplantation.

In certain embodiments, defibrotide is administered prophylactically. In some embodiments, the one or more administrations of the defibrotide begins before the patient starts a conditioning regimen for hematopoietic stem cell transplantation. In some embodiments, the one or more administrations of the defibrotide begins the day before the patient starts a conditioning regimen for hematopoietic stem cell transplantation. Thus, in some embodiments, the administrations of the defibrotide begin before or on Day 0 to Day +30 of the pre-HSCT conditioning regimen. In some embodiments, the administrations of the defibrotide begin before or on Day −6 to Day +24 of the HSCT transplant regimen. Thus, in some embodiments, the administration of the defibrotide begins 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days before the patient starts a conditioning regimen for hematopoietic stem cell transplantation. In some embodiments, the administration of the defibrotide begins one day before the patient starts a conditioning regimen for hematopoietic stem cell transplantation and continues for at least 21 days. In some embodiments, the administration of the defibrotide begins one day before the patient starts a conditioning regimen for hematopoietic stem cell transplantation (e.g. 6 days before hematopoietic stem cell transplantation) and continues for at least 15 days after HSCT transplantation. Thus in some embodiments, the administrations starts one day before the patient starts a conditioning regimen and continues for 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

In accordance with some embodiments of the present disclosure, the patient is from about 0 years of age to about 16 years of age, including all ranges and subranges therein. For example, the patient is from about 0 months, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, to about 16 years of age. In some embodiments, the patient is from about 0 months to about 23 months of age. In some embodiments, the patient is from about 2 years to about 11 years of age. In some embodiments, the patient is from about 12 years to about 16 years of age.

In accordance with some embodiments of the present disclosure, the patient may be a pediatric patient or adult. A pediatric patient is from about 0 years of age to about 16 years of age, including all ranges and subranges therein. For example, the pediatric patient is from about 0 months, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, to about 16 years of age. In some embodiments, the patient is from about 0 months to about 23 months of age. In some embodiments, the patient is from about 2 years to about 11 years of age. In some embodiments, the patient is from about 12 years to about 16 years of age. An adult patient is older than 16 years of age.

The patient of the present disclosure may have a variety of underlying primary diseases. Examples of primary diseases the patient of the present disclosure may have, include but are not limited to: acute myelogenous leukemia, acute lymphocytic leukemia, neuroblastoma, myelodysplastic syndrome, non-Hodgkin's lymphoma, Hodgkin's lymphoma, chronic myelogenous leukemia, multiple myeloma, chronic lymphocytic leukemia, other leukemia, or congenital neutropenia. Thus, in some embodiments, the patient of the present disclosure has acute myelogenous leukemia as a primary disease. In some embodiments, the patient of the present disclosure has acute lymphocytic leukemia as a primary disease. In some embodiments, the patient of the present disclosure has neuroblastoma as a primary disease. In some embodiments, the patient of the present disclosure has myelodysplastic syndrome as a primary disease. In some embodiments, the patient of the present disclosure has non-Hodgkin's lymphoma as a primary disease. In some embodiments, the patient of the present disclosure has Hodgkin's lymphoma as a primary disease. In some embodiments, the patient of the present disclosure has chronic myelogenous leukemia as a primary disease. In some embodiments, the patient of the present disclosure has multiple myeloma as a primary disease. In some embodiments, the patient of the present disclosure has chronic lymphocytic leukemia as a primary disease. In some embodiments, the patient of the present disclosure has congenital neutropenia as a primary disease.

In certain embodiments, patients having undergone stem cell transplantation may have undergone allogenic or autologous stem cell transplantation. In some embodiments, the patient of the present disclosure has undergone allogenic stem cell transplantation. In some embodiments, the patient of the present disclosure has undergone autologous stem cell transplantation.

In certain embodiments, patients being prepared for stem cell transplantation may be prepared for allogenic or autologous stem cell transplantation. In some embodiments, the patient of the present disclosure is being prepared for allogenic stem cell transplantation. In some embodiments, the patient of the present disclosure is being prepared for autologous stem cell transplantation.

Graft-versus-host disease (GVHD) associated with stem cell transplantation (e.g., allogenic) is a serious life-threatening complication. Thus, patients may or may not undergo a prophylactic regimen to prevent or decrease the severity of GVHD. Examples of therapeutic agents which may be taken for GVHD prophylaxis include, but are not limited to heparin, tacrolimus, methotrexate, cyclosporine, sirolimus, or a combination hereof. In some embodiments, the patient of the present disclosure does not take a therapeutic agent for GVHD prophylaxis. In some embodiments, the patient of the present disclosure takes tacrolimus for GVHD prophylaxis. In some embodiments, the patient of the present disclosure takes methotrexate for GVHD prophylaxis. In some embodiments, the patient of the present disclosure takes cyclosporine for GVHD prophylaxis. In some embodiments, the patient of the present disclosure takes sirolimus for GVHD prophylaxis.

In some embodiments, the present disclosure provides a method for treating a patient diagnosed as having sinusoidal obstruction syndrome, comprising administering a therapeutically effective amount of defibrotide to the patient, where the patient has undergone at least one chemotherapy treatment and has not undergone hematopoietic stem cell transplantation, and where one or more administrations of the defibrotide are given to the patient over a treatment period. The patient having sinusoidal obstruction syndrome may or may not have multiple organ dysfunction. Thus, in some embodiments, the patient has multiple organ dysfunction. In some embodiments, the patient does not have multiple organ dysfunction.

In some embodiments, the one or more defibrotide treatments may begin before the patient is diagnosed with SOS. In some embodiments, the one or more defibrotide treatments may begin on the same day as the patient was diagnosed with SOS or, for a variety of reason which are readily apparent to a skilled artisan, they may begin on a day after the patient was diagnosed with SOS. For example, the defibrotide treatments may begin on days 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 after the patient was diagnosed as having SOS. Thus, in some embodiments, the one or more administrations of the defibrotide begins on the same day that the patient diagnosed as having sinusoidal obstruction syndrome (i.e., day 0). In other embodiment, the one or more administrations of the defibrotide begins on 1, 2, 3, 4, 5, 6, or 7 days after the patient diagnosed as having sinusoidal obstruction syndrome. In some embodiments, the one or more administrations of defibrotide begin 1 day after the patient was diagnosed as having SOS. In some embodiments, the one or more administrations of defibrotide begin 2 days after the patient was diagnosed as having SOS. In some embodiments, the one or more administrations of defibrotide begin 3 days after the patient was diagnosed as having SOS. In some embodiments, the one or more administrations of defibrotide begin 4 days after the patient was diagnosed as having SOS. In some embodiments, the one or more administrations of defibrotide begin 5 days after the patient was diagnosed as having SOS. In some embodiments, the one or more administrations of defibrotide begin 6 days after the patient was diagnosed as having SOS. In some embodiments, the one or more administrations of defibrotide begin 7 days after the patient was diagnosed as having SOS.

As a skilled artisan will appreciate, the defibrotide treatment period may vary on a patient-by-patient basis. In some embodiments, the defibrotide treatment period is determined by monitoring signs and symptoms of hepatic VOD. For example, if the signs and symptoms of hepatic VOD are still present after an initial treatment period, defibrotide treatment is continued until resolution of VOD. In some embodiments, if the signs and symptoms of hepatic VOD are still present after 21 days of treatment, defibrotide treatment is continued until resolution of VOD up to a maximum of 60 days. Thus, in certain embodiments, the treatment period may last anywhere from 21 to 60 days. For example, the treatment period lasts for 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days. In some embodiments, the treatment period lasts 21 days.

The defibrotide dosing may be determined by a variety of factors that will be readily apparent to a skilled artisan. In some embodiments, the dose is based on patient's baseline body weight, defined as the patient's weight prior to the preparative regimen for HSCT. In some embodiments, defibrotide is administered in an amount of about 1 to about 100 mg/kg of body weight per day. For example the defibrotide is administered in an amount of about 1 mg/kg, about 1.25 mg/kg, about 1.50 mg/kg, about 1.75 mg/kg, about 2 mg/kg, about 2.25 mg/kg, about 2.50 mg/kg, about 2.75 mg/kg, about 3 mg/kg, about 3.25 mg/kg, about 3.50 mg/kg, about 3.75 mg/kg, about 4.25 mg/kg, about 4.50 mg/kg, about 4.75 mg/kg, about 5 mg/kg, about 5.25 mg/kg, about 5.50 mg/kg, about 5.75 mg/kg, about 6 mg/kg, about 6.25 mg/kg, about 6.50 mg/kg, about 6.75 mg/kg, about 7 mg/kg, about 7.25 mg/kg, about 7.50 mg/kg, about 7.75 mg/kg, about 8 mg/kg, about 8.25 mg/kg, about 8.50 mg/kg, about 8.75 mg/kg, about 9 mg/kg, about 9.25 mg/kg, about 9.50 mg/kg, about 9.75 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg of body weight per day, including all ranges therebetween. In some embodiments, defibrotide is administered in an amount of about 25 mg per kilogram of body weight per day. In some embodiments, doses based on the patient's body weight were rounded to the nearest 10 mg for patients over 35 kg. In some embodiments, doses based on the patient's body weight were rounded to the nearest 5 mg for patients under 35 kg. In some embodiments, the defibrotide is administered as a high concentration low viscosity formulation, as described in WO 2019/028340 the contents of which are incorporated by reference in their entirety for all purposes.

The defibrotide may be administered as a single daily dose or in multiple doses per day. In some embodiments, defibrotide is administered once a day. In some embodiments, defibrotide is administered in multiple doses per day. For example, defibrotide may be administered in 2, 3, 4, 5, 6, 7, 8, 9, or in 10 doses per day. In some embodiments, defibrotide is administered in four doses per day. In some embodiments, defibrotide is administered in four doses per day every 6 hours.

The defibrotide may be administered by any suitable route, including without limitation parenteral (e.g., intravenous, subcutaneous, intrasternal, intramuscular, or infusion techniques), oral, sublingual, buccal, intranasal, pulmonary, topical, transdermal, intradermal, mucosal, ocular, otic, rectal, vaginal, intragastric, intrasynovial, and intra-articular routes. In some embodiments, defibrotide is administered intravenously. In some embodiments, defibrotide is administered via intravenous infusion. In some embodiments, defibrotide is administered by constant intravenous infusion over a 2-hour period. In some embodiments, the defibrotide is diluted prior to infusion. In some embodiments, the diluted defibrotide solution is administered using an infusion set equipped with a filter (e.g., a 0.2 micron in-line filter). In certain embodiments, the intravenous administration line (e.g., peripheral or central) is flushed immediately before and after administration (e.g., with 5% Dextrose Injection, USP or 0.9% Sodium Chloride Injection, USP).

Any combination of the foregoing embodiments may be used in treating the patient with defibrotide. Accordingly, in some embodiments, the patient is administered defibrotide intravenously in an amount of about 6.25 mg per kilogram of body weight about every 6 hours. In some embodiments, the patient is administered defibrotide intravenously in an amount of about 6.25 mg per kilogram of body weight about every 6 hours given as a 2-hour intravenous infusion. In some embodiments, the patient is administered defibrotide intravenously in an amount of about 6.25 mg per kilogram of body weight about every 6 hours given as a constant 2-hour intravenous infusion.

In some embodiments, administration of defibrotide treats or ameliorates development of SOS and/or SOS symptoms compared to an untreated patient or the same patient before defibrotide administration. In some embodiments, SOS and/or SOS symptoms are treated or ameliorated in the patient between day 1 and year 10. In some embodiments, administration of defibrotide treats or ameliorates development of SOS and/or SOS symptoms compared to an untreated patient or the same patient before defibrotide administration at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, administration of defibrotide treats or ameliorates development of SOS and/or SOS symptoms compared to an untreated patient or the same patient before defibrotide administration for about 1 day, about 1 week, about 1 month, about 2 months, about 3 months, about 4 months, about 5 months, about 6 months, about 1 year, about 2 years, about 5 years, or about 10 years, or more.

In some embodiments, administration of defibrotide treats or ameliorates SOS and/or SOS symptoms by about 1%, about 5%, about 10%, about 20%, about 30%, about 40%, about 50%, about 60%, about 70%, about 80%, about 90%, or about 100% compared to an untreated patient or the same patient before defibrotide administration. In some embodiments, administration of defibrotide treats or ameliorates development of SOS and/or SOS symptoms compared to an untreated patient or the same patient before defibrotide administration by about 1%, about 5%, about 10%, about 20%, about 30%, about 40%, about 50%, about 60%, about 70%, about 80%, about 90%, or about 100% at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, administration of defibrotide treats or ameliorates development of SOS and/or SOS symptoms compared to an untreated patient or the same patient before defibrotide administration by about 1%, about 5%, about 10%, about 20%, about 30%, about 40%, about 50%, about 60%, about 70%, about 80%, about 90%, or about 100% for about 1, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more.

In some embodiments, prophylactic administration of defibrotide prevents, delays, or ameliorates development of SOS and/or SOS symptoms compared to an untreated patient or a patient treated after HSCT conditioning begins. In some embodiments, SOS and/or SOS symptoms are prevented in the prophylactically-treated patient between day 1 and year 10. In some embodiments, prophylactic administration of defibrotide prevents, delays, or ameliorates development of SOS and/or SOS symptoms compared to an untreated patient or a patient treated with defibrotide after HSCT conditioning begins at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, prophylactic administration of defibrotide prevents, delays, or ameliorates development of SOS and/or SOS symptoms compared to an untreated patient or a patient treated with defibrotide after HSCT conditioning begins for about 1 day, about 1 week, about 1 month, about 2 months, about 3 months, about 4 months, about 5 months, about 6 months, about 1 year, about 2 years, about 5 years, or about 10 years, or more.

In some embodiments, prophylactic administration of defibrotide prevents, delays, or ameliorates SOS and/or SOS symptoms by about 1%, about 5%, about 10%, about 20%, about 30%, about 40%, about 50%, about 60%, about 70%, about 80%, about 90%, or about 100% compared with untreated patients or patients treated with defibrotide after HSCT conditioning begins. In some embodiments, prophylactic administration of defibrotide prevents, delays, or ameliorates development of SOS and/or SOS symptoms compared to an untreated patient or a patient treated with defibrotide after HSCT conditioning begins by about 1%, about 5%, about 10%, about 20%, about 30%, about 40%, about 50%, about 60%, about 70%, about 80%, about 90%, or about 100% at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, prophylactic administration of defibrotide prevents, delays, or ameliorates development of SOS and/or SOS symptoms compared to an untreated patient or a patient treated with defibrotide after HSCT conditioning begins by about 1%, about 5%, about 10%, about 20%, about 30%, about 40%, about 50%, about 60%, about 70%, about 80%, about 90%, or about 100% for about 1, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more.

In some embodiments, prophylactic administration of defibrotide prevents, delays, or ameliorates development of GVHD and/or GVHD symptoms compared to an untreated patient or a patient treated with defibrotide after HSCT conditioning begins. In some embodiments, prophylactic administration of defibrotide prevents, delays, or ameliorates development of GVHD and/or GVHD symptoms compared to an untreated patient or a patient treated with defibrotide after HSCT conditioning begins between day 1 and year 10. In some embodiments, prophylactic administration of defibrotide prevents, delays, or ameliorates development of GVHD and/or GVHD symptoms compared to an untreated patient or a patient treated with defibrotide after HSCT conditioning begins at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, prophylactic administration of defibrotide prevents, delays, or ameliorates development of GVHD and/or GVHD symptoms compared to an untreated patient or a patient treated with defibrotide after HSCT conditioning begins for about 1 day, 2 days, 3 days, 4 days, 5 days, 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years, or more.

In some embodiments, prophylactic administration of defibrotide prevents, delays, or ameliorates GVHD and/or GVHD symptoms by about 1%, about 5%, about 10%, about 20%, about 30%, about 40%, about 50%, about 60%, about 70%, about 80%, about 90%, or about 100% compared with untreated patients or patients treated with defibrotide after HSCT conditioning begins. In some embodiments, prophylactic administration of defibrotide prevents, delays, or ameliorates development of GVHD and/or GVHD symptoms compared to an untreated patient or a patient treated with defibrotide after HSCT conditioning begins by about 1%, about 5%, about 10%, about 20%, about 30%, about 40%, about 50%, about 60%, about 70%, about 80%, about 90%, or about 100% at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, prophylactic administration of defibrotide prevents, delays, or ameliorates development of GVHD and/or GVHD symptoms compared to an untreated patient or a patient treated with defibrotide after HSCT conditioning begins by about 1%, about 5%, about 10%, about 20%, about 30%, about 40%, about 50%, about 60%, about 70%, about 80%, about 90%, or about 100% for about 1, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more.

The following examples further illustrate various embodiments of the present disclosure, but should not be construed in any way as limiting its scope.

### EXAMPLES

### Example 1

### Determination of Efficacy and Safety for Patients Treated with Defibrotide Having Late-Onset SOS Following HSCT

Study Design. A multicenter, single-arm, open-label, T-IND study designed to provide expanded access to defibrotide was conducted to assess safety and survival in patients with SOS (e.g., hepatic SOS), with or without MOD. Adults and pediatric patients with SOS after HSCT or chemotherapy alone (i.e., without HSCT) were tested (ClinicalTrials.gov Registry No.: NCT00628498). The primary efficacy measure was the day +100 survival after HSCT or initiation of primary chemotherapy. Safety endpoints included treatment-emergent adverse events (TEAEs) and treatment-related adverse events (TRAEs).

The T-IND included a total of 1154 patients enrolled at 101 centers across the United States who received at least 1 dose of defibrotide following HSCT or chemotherapy without HSCT (safety population). Among the 1149 patients from the safety population with available duration of treatment data, the mean (standard deviation) and median length of defibrotide treatment was 21.5 (13.51) and 21.0 days, respectively. Of those enrolled, 1137 met criteria for SOS (SOS efficacy population). The Kaplan-Meier estimated Day +100 survival rate for the efficacy population was 61.1% (95% CI, 58.2%-63.9%) and for the 571 patients with MOD was 51.9% (95% CI, 47.6%-55.9%). TRAEs were reported in 21.5% of the total population (N=1154). The most common TRAEs were pulmonary hemorrhage, (4.3%), gastrointestinal hemorrhage (3.0%), epistaxis (2.3%), and hypotension (2.1%). Serious TRAEs were reported in 11.5% of patients. TRAEs led to discontinuation or death in 12.0% and 2.7% of patients, respectively. The detailed analysis of the nontransplant-associated VOD/SOS subgroup is reported in Examples 3-4. All subsequent results reported in Examples 1-2 are from the analysis of the patient population diagnosed with SOS following HSCT (post-HSCT population) (n=1000).

Assessments. Day +100 survival was assessed via Kaplan-Meier estimate with 95% confidence interval (CI). Safety data included new or worsening adverse events (AEs) and symptoms of the original or targeted disease were not to be reported as AEs unless the event was considered serious.

Patients. Eligibility for the T-IND study originally required SOS diagnosed by the Baltimore criteria or biopsy by Day +35 after HSCT with MOD (renal dysfunction or pulmonary dysfunction) by Day +45 and have provided written informed consent. Pulmonary dysfunction was defined as supplemental oxygen, oxygen saturation ≤90% on room air, or ventilator dependence. Renal dysfunction was defined as creatinine clearance or glomeruli filtration rate ≤40% of baseline, serum creatinine ≥3× baseline value, or dialysis dependence.

The protocol was later amended to also include patients with: late-onset SOS, SOS without MOD, SOS diagnosed by modified Seattle criteria (using ≥5% weight-gain threshold), and SOS after chemotherapy without HSCT. In this analysis, late-onset SOS was defined as diagnosis occurring >21 days post-HSCT. Hemodynamic/ultrasound data, required by the EBMT adult criteria for diagnosis of SOS beyond day 21, were not available for the patients in this study. Key exclusion criteria included acute bleeding or hemodynamic instability (i.e., need for ≥2 vasopressors), medication that increased risk of bleeding, or pregnancy.

Among all 570 pediatric patients in the general overall HSCT population, 28.2% were aged 0-23 months, 52.5% were aged 2-11 years, and 19.3% were aged 12-16 years. By age subgroup, acute myelogenous leukemia was more common in adults (34.7%) than children (19.6%). In contrast, neuroblastoma was the underlying disease for HSCT more commonly in children (18.2%) than adults (0.2%). Acute lymphocytic leukemia was common in both adult (22.1%) and pediatric patients (18.1%).

Dosing. Defibrotide was administered intravenously 4 times daily (6.25 mg/kg in 2-hour infusions at 6-hour intervals; total 25 mg/kg/day), with a recommended duration of 21 days or more. Doses may have been held to accommodate other procedures (e.g., intermittent hemodialysis or surgery).

### Example 2

### Results and Discussion of Defibrotide Administration to Patients Having Late-Onset SOS Following HSCT

1000 patients who had confirmed SOS post-HSCT were treated with defibrotide on the protocol described above. Demographics and disease characteristics of the overall T-IND patient groups having SOS after HSCT, with or without MOD are presented in Table 1. Of the final T-IND patient data for patients with SOS after HSCT who received defibrotide (n=1000), 264 patients (26.4%) were diagnosed with late-onset SOS post-HSCT. The late-onset baseline demographics are shown in Table 2. The median age of the late-onset cohort was 27 years, and 64.0% of patients were adults (>16 years), which was substantially older than the overall HSCT population from the T-IND, which had a median age of 14 years. The most common primary diseases in the late-onset cohort were acute leukemias (55.2%) and most patients received allogeneic transplants (86.0%) (89.9% in the subgroup with MOD, and 81.6% in the subgroup without MOD). Use of tacrolimus was higher in the late-onset group (61.7%) compared with the group with onset ≤21-days (43.5%); The most common graft-vs-host disease (GVHD) prophylaxis in the late-onset group were regimens containing tacrolimus (61.7%), methotrexate (31.4%), cyclosporine (16.3%), and sirolimus (14.8%). Diagnosis of VOD/SOS after Day +100 occurred in 1.1% of patients, and the median day of late-onset was 29 (range, 22 to 335).

TABLE 1
Baseline Demographics and Disease Characteristics of the HSCT Population

| | MOD | | | | | | No MOD | | | |
|---|---|---|---|---|---|---|---|---|---|---|
| | Overall | | >16 years | | ≤16 years | | Overall | | >16 years | |
| Characteristic | (n = 512) | | (n = 231) | | (n = 281) | | (n = 488) | | (n = 199) | |
| Sex, n | | | | | | | | | | |
| Male | 278 | (54.3%) | 119 | (51.5%) | 159 | (56.6%) | 290 | (59.4%) | 118 | (59.3%) |
| Female | 234 | (45.7%) | 112 | (48.5%) | 122 | (43.4%) | 198 | (40.6%) | 81 | (40.7%) |
| Race | | | | | | | | | | |
| White | 343 | (67.0%) | 175 | (75.8%) | 168 | (59.8%) | 284 | (58.2%) | 135 | (67.8%) |
| African American | 36 | (7.0%) | 13 | (5.6%) | 23 | (8.2%) | 44 | (9.0%) | 16 | (8.0%) |
| Asian | 20 | (3.9%) | 9 | (3.9%) | 11 | (3.9%) | 34 | (7.0%) | 13 | (6.5%) |
| Latino | 71 | (13.9%) | 23 | (10.0%) | 48 | (17.1%) | 92 | (18.9%) | 27 | (13.6%) |
| Age at time of HSCT, years, | 14.00 | (0.10, 69.00) | 41.00 | (17.00, 69.00) | 4.00 | (0.10, 16.00) | 12.00 | (0.10, 77.00) | 43.00 | (17.00, 77.00) |
| median (range) | | | | | | | | | | |
| Conditioning agents (>10%), n (%) | | | | | | | | | | |
| Cyclophosphamide | 333 | (65.0) | 154 | (66.7) | 179 | (63.7) | 241 | (49.4) | 93 | (46.7) |
| Busulfan | 262 | (51.2) | 100 | (43.3) | 162 | (57.7) | 301 | (61.7) | 101 | (50.8) |
| Fludarabine | 165 | (32.2) | 88 | (38.1) | 77 | (27.4) | 177 | (36.3) | 97 | (48.7) |
| Anti-thymocyte globulin | 154 | (30.1) | 35 | (15.2) | 119 | (42.3) | 152 | (31.1) | 41 | (20.6) |
| Total body irradiation | 172 | (33.6) | 104 | (45.0) | 68 | (24.2) | 115 | (23.6) | 66 | (33.2) |
| Melphalan | 117 | (22.9) | 30 | (13.0) | 87 | (31.0) | 142 | (29.1) | 38 | (19.1) |
| Thiopeta | 64 | (12.5) | 26 | (11.3) | 38 | (13.5) | 70 | (14.3) | 20 | (10.1) |
| GVHD prophylaxis, n (%) | | | | | | | | | | |
| Tacrolimus | 250 | (48.8%) | 162 | (70.1%) | 88 | (31.3%) | 233 | (47.7%) | 137 | (68.8%) |
| Methotrexate | 168 | (32.8%) | 101 | (43.7%) | 67 | (23.8%) | 164 | (33.6%) | 82 | (41.2%) |
| Cyclosporine | 159 | (31.1%) | 38 | (16.5%) | 121 | (43.1%) | 123 | (25.2%) | 26 | (13.1%) |
| Sirolimus | 59 | (11.5%) | 47 | (20.3%) | 12 | (4.3%) | 30 | (6.1%) | 26 | (13.1%) |
| None | 73 | (14.3%) | 14 | (6.1%) | 59 | (21.0%) | 108 | (22.1%) | 24 | (12.1%) |
| Other^{a} | 182 | (35.5%) | 74 | (32.0%) | 108 | (38.4%) | 160 | (32.8%) | 66 | (33.2%) |
| Transplant type^{b} | | | | | | | | | | |
| Allogeneic | 450 | (87.9%) | 220 | (95.2) | 230 | (81.9) | 393 | (80.5%) | 182 | (91.5) |
| Autologous | 61 | (11.9%) | 11 | (4.8) | 50 | (17.8) | 94 | (19.3%) | 17 | (8.5) |
| Primary disease (>10%), n (%) | | | | | | | | | | |
| AML | 142 | (27.7%) | 88 | (38.1%) | 54 | (19.2%) | 119 | (24.4%) | 61 | (30.7%) |
| ALL | 111 | (21.7%) | 48 | (20.8%) | 63 | (22.4%) | 90 | (18.4%) | 49 | (24.6%) |
| Neuroblastoma | 43 | (8.4%) | 1 | (0.4%) | 42 | (14.9%) | 62 | (12.7%) | 0 | (0%) |
| Prior HSCTs, n (%) | | | | | | | | | | |
| 0 | 428 | (83.6) | 189 | (81.8) | 239 | (85.1) | 401 | (82.2) | 162 | (81.4) |
| ≥1 | 82 | (16.0) | 41 | (17.7) | 41 | (14.6) | 83 | (17.0) | 35 | (17.6) |
| Unknown/Missing | 2 | (0.4) | 1 | (0.4) | 1 | (0.4) | 4 | (0.8) | 2 | (1.0) |
| VOD/SOS onset after day 21, | 139 | (27.1) | 88 | (38.1) | 51 | (18.1) | 125 | (25.6) | 81 | (40.7) |
| n (%) | | | | | | | | | | |
| Time from diagnosis to start of | | | | | | | | | | |
| defibrotide, n (%) | | | | | | | | | | |
| Day of diagnosis | 156 | (30.4) | 68 | (29.4) | 88 | (31.3) | 154 | (31.6) | 52 | (26.1) |
| Day 1 | 135 | (26.4) | 63 | (27.3) | 72 | (25.6) | 151 | (30.9) | 49 | (24.6) |
| Day 2 | 68 | (13.3) | 27 | (11.7) | 41 | (14.6) | 57 | (11.7) | 26 | (13.1) |
| Day 3 | 39 | (7.6) | 16 | (6.9) | 23 | (8.2) | 38 | (7.8) | 20 | (10.1) |
| Day 4 | 33 | (6.4) | 15 | (6.5) | 18 | (6.4) | 28 | (5.7) | 12 | (6.0) |
| Day 5 | 17 | (3.3) | 10 | (4.3) | 7 | (2.5) | 19 | (3.9) | 12 | (6.0) |
| Day 6 | 9 | (1.8) | 6 | (2.6) | 3 | (1.1) | 6 | (1.2) | 3 | (1.5) |
| Day 7 | 12 | (2.3) | 5 | (2.2) | 7 | (2.5) | 7 | (1.4) | 4 | (2.0) |
| Days 8-14 | 29 | (5.7) | 15 | (6.5) | 14 | (4.9) | 16 | (3.3) | 11 | (5.5) |
| Day ≥15 | 14 | (2.7) | 6 | (2.6) | 8 | (2.8) | 12 | (2.5) | 10 | (5.0) |

| | No MOD | | All HSCT | | | | | |
|---|---|---|---|---|---|---|---|---|
| | ≤16 years | | Overall | | >16 years | | ≤16 years | |
| Characteristic | (n = 289) | | (n = 1000) | | (n = 430) | | (n = 570) | |
| Sex, n | | | | | | | | |
| Male | 172 | (59.5%) | 568 | (56.8%) | 237 | (55.1%) | 331 | (58.1%) |
| Female | 117 | (40.5%) | 432 | (43.2%) | 193 | (44.9%) | 239 | (41.9%) |
| Race | | | | | | | | |
| White | 149 | (51.6%) | 627 | (62.7%) | 310 | (72.1%) | 317 | (55.6%) |
| African American | 28 | (9.7%) | 80 | (8.0%) | 29 | (6.7%) | 51 | (8.9%) |
| Asian | 21 | (7.3%) | 54 | (5.4%) | 22 | (5.1%) | 32 | (5.6%) |
| Latino | 65 | (22.5%) | 163 | (16.3%) | 50 | (11.6%) | 113 | (19.8%) |
| Age at time of HSCT, years, | 3.00 | (0.10, 16.00) | 14.00 | (0.10, 77.00) | 42.00 | (17.00, 77.00) | 4.00 | (0.10, 16.00) |
| median (range) | | | | | | | | |
| Conditioning agents (>10%), n (%) | | | | | | | | |
| Cyclophosphamide | 148 | (51.2) | 574 | (57.4) | 247 | (57.4) | 327 | (57.4) |
| Busulfan | 200 | (69.2) | 563 | (56.3) | 201 | (46.7) | 362 | (63.5) |
| Fludarabine | 80 | (27.7) | 342 | (34.2) | 185 | (43.0) | 157 | (27.5) |
| Anti-thymocyte globulin | 111 | (38.4) | 306 | (30.6) | 76 | (17.7) | 230 | (40.4) |
| Total body irradiation | 49 | (17.0) | 287 | (28.7) | 170 | (39.5) | 117 | (20.5) |
| Melphalan | 104 | (36.0) | 259 | (25.9) | 68 | (15.8) | 191 | (33.5) |
| Thiopeta | 50 | (17.3) | 134 | (13.4) | 46 | (10.7) | 88 | (15.4) |
| GVHD prophylaxis, n (%) | | | | | | | | |
| Tacrolimus | 96 | (33.2%) | 483 | (48.3%) | 299 | (69.5%) | 184 | (32.3%) |
| Methotrexate | 82 | (28.4%) | 332 | (33.2%) | 183 | (42.6%) | 149 | (26.1%) |
| Cyclosporine | 97 | (33.6%) | 282 | (28.2%) | 64 | (14.9%) | 218 | (38.2%) |
| Sirolimus | 4 | (1.4%) | 89 | (8.9%) | 73 | (17.0%) | 16 | (2.8%) |
| None | 84 | (29.1%) | 181 | (18.1%) | 38 | (8.8%) | 143 | (25.1%) |
| Other^{a} | 94 | (32.5%) | 342 | (34.2%) | 140 | (32.6%) | 202 | (35.4%) |
| Transplant type^{b} | | | | | | | | |
| Allogeneic | 211 | (73.0) | 843 | (84.3%) | 402 | (93.5) | 441 | (77.4) |
| Autologous | 77 | (26.6) | 155 | (15.5%) | 28 | (6.5) | 127 | (22.3) |
| Primary disease (>10%), n (%) | | | | | | | | |
| AML | 58 | (20.1%) | 261 | (26.1%) | 149 | (34.7%) | 112 | (19.6%) |
| ALL | 41 | (14.2%) | 201 | (20.1%) | 97 | (22.6%) | 104 | (18.2%) |
| Neuroblastoma | 62 | (21.5%) | 105 | (10.5%) | 1 | (0.2%) | 104 | (18.2%) |
| Prior HSCTs, n (%) | | | | | | | | |
| 0 | 239 | (82.7) | 829 | (82.9) | 351 | (81.6) | 478 | (83.9) |
| ≥1 | 48 | (16.6) | 165 | (16.5) | 76 | (17.7) | 89 | (15.6) |
| Unknown/Missing | 2 | (0.7) | 6 | (0.6) | 3 | (0.7) | 3 | (0.5) |
| VOD/SOS onset after day 21, | 44 | (15.2) | 264 | (26.4) | 169 | (39.3) | 95 | (16.7) |
| n (%) | | | | | | | | |
| Time from diagnosis to start of | | | | | | | | |
| defibrotide, n (%) | | | | | | | | |
| Day of diagnosis | 102 | (35.3) | 310 | (31.0) | 120 | (27.9) | 190 | (33.3) |
| Day 1 | 102 | (35.3) | 286 | (28.6) | 112 | (26.0) | 174 | (30.5) |
| Day 2 | 31 | (10.7) | 125 | (12.5) | 53 | (12.3) | 72 | (12.6) |
| Day 3 | 18 | (6.2) | 77 | (7.7) | 36 | (8.4) | 41 | (7.2) |
| Day 4 | 16 | (5.5) | 61 | (6.1) | 27 | (6.3) | 34 | (5.9) |
| Day 5 | 7 | (2.4) | 36 | (3.6) | 22 | (5.1) | 14 | (2.5) |
| Day 6 | 3 | (1.0) | 15 | (1.5) | 9 | (2.1) | 6 | (1.1) |
| Day 7 | 3 | (1.0) | 19 | (1.9) | 9 | (2.1) | 10 | (1.8) |
| Days 8-14 | 5 | (1.7) | 45 | (4.5) | 29 | (6.7) | 19 | (3.3) |
| Day ≥15 | 2 | (0.7) | 26 | (2.6) | 16 | (3.7) | 10 | (1.8) |

^{a}Includes medications that were not prespecified on the case report form;
ALL, acute lymphocytic leukemia; AML, acute myelogenous leukemia;

TABLE 2
Baseline Demographics and Disease Characteristics for Late-Onset Cohort

| | All Late Onset SOS | | | | | | SOS with MOD | | | |
|---|---|---|---|---|---|---|---|---|---|---|
| | Overall | | >16 years | | ≤16 years | | Overall | | >16 years | |
| | (n = 264) | | (n = 169) | | (n = 95) | | (n = 139) | | (n = 88) | |
| Sex | | | | | | | | | | |
| Male | 133 | (50.4) | 83 | (49.1) | 50 | (52.6) | 70 | (50.4) | 44 | (50.0) |
| Female | 131 | (49.6) | 86 | (50.9) | 45 | (47.4) | 69 | (49.6) | 44 | (50.0) |
| Race | | | | | | | | | | |
| White | 167 | (63.3) | 117 | (69.2) | 50 | (52.6) | 87 | (62.6) | 63 | (71.6) |
| Non-white | 97 | (36.7) | 52 | (30.8) | 45 | (47.4) | 52 | (37.4) | 25 | (38.4) |
| Median age at HSCT, years | 27.0 | | 45.0 | | 5.0 | | 24.0 | | 43.5 | |
| Age class at HSCT | 169 | (64.0) | 169 | (100) | — | | 88 | (63.3) | 88 | (100) |
| >16 years | 95 | (36.0) | — | | 95 | (100) | 51 | (36.7) | — | |
| ≤16 years | 15 | (5.7) | — | | 15 | (15.8) | 6 | (4.3) | — | |
| 0-23 | 60 | (22.7) | — | | 60 | (63.2) | 35 | (25.2) | — | |
| months | 20 | (7.6) | — | | 20 | (21.1) | 10 | (7.2) | — | |
| Primary disease^{a} (>5%) | | | | | | | | | | |
| Acute myelogenous leukemia | 94 | (36.0) | 76 | (45.5) | 18 | (19.1) | 56 | (40.9) | 44 | (50.6) |
| Acute lymphocytic leukemia | 50 | (19.2) | 29 | (17.4) | 21 | (22.3) | 29 | (21.2) | 15 | (17.2) |
| Neuroblastoma | 24 | (9.2) | — | | 24 | (25.5) | 8 | (5.8) | — | |
| Myelodysplastic syndrome | 12 | (4.6) | 8 | (4.8) | 4 | (4.3) | 4 | (2.9) | 4 | (4.6) |
| Non-Hodgkin lymphoma | 12 | (4.6) | 11 | (6.6) | 1 | (1.1) | 5 | (3.6) | 4 | (4.6) |
| Other | 35 | (13.4) | 17 | (10.2) | 18 | (19.1) | 20 | (14.6) | 11 | (12.6) |
| Type of transplant | | | | | | | | | | |
| Allogeneic | 227 | (86.0) | 158 | (93.5) | 69 | (72.6) | 125 | (89.9) | 84 | (95.5) |
| Autologous | 37 | (14.0) | 11 | (6.5) | 26 | (27.4) | 14 | (10.1) | 4 | (4.5) |
| GVHD prophylaxis | | | | | | | | | | |
| None | 44 | (16.7) | 14 | (8.3) | 30 | (31.6) | 16 | (11.5) | 14 | (8.3) |
| Tacrolimus | 163 | (61.7) | 128 | (75.7) | 35 | (36.8) | 92 | (66.2) | 128 | (75.7) |
| Methotrexate | 83 | (31.4) | 66 | (39.1) | 17 | (17.9) | 45 | (32.4) | 66 | (39.1) |
| Cyclosporine | 43 | (16.3) | 17 | (10.1) | 26 | (27.4) | 24 | (17.3) | 17 | (10.1) |
| Sirolimus | 39 | (14.8) | 33 | (19.5) | 6 | (6.3) | 27 | (19.4) | 33 | (19.5) |
| Other | 85 | (32.2) | 53 | (31.4) | 32 | (33.7) | 51 | (36.7) | 53 | (31.4) |

| | SOS with MOD | | SOS without MOD | | | | | |
|---|---|---|---|---|---|---|---|---|
| | ≤16 years | | Overall | | >16 years | | ≤16 years | |
| | (n = 51) | | (n = 125) | | (n = 81) | | (n = 44) | |
| Sex | | | | | | | | |
| Male | 26 | (51.0) | 63 | (50.4) | 39 | (48.1) | 24 | (54.4) |
| Female | 25 | (49.0) | 62 | (49.6) | 42 | (51.9) | 20 | (49.0) |
| Race | | | | | | | | |
| White | 24 | (47.1) | 80 | (64.0) | 54 | (66.7) | 26 | (59.1) |
| Non-white | 27 | (52.9) | 45 | (36.0) | 27 | (33.3) | 18 | (40.9) |
| Median age at HSCT, years | 5.0 | | 33.0 | | 47.0 | | 5.0 | |
| Age class at HSCT | — | | 81 | (64.8) | 81 | (100) | — | |
| >16 years | 51 | (100) | 44 | (35.2) | — | | 44 | (100) |
| ≤16 years | 6 | (11.8) | 9 | (7.2) | — | | 9 | (20.5) |
| 0-23 | 35 | (68.6) | 25 | (20.0) | — | | 25 | (56.8) |
| months | 10 | (19.6) | 10 | (8.0) | — | | 10 | (22.7) |
| Primary disease^{a} (>5%) | | | | | | | | |
| Acute myelogenous leukemia | 12 | (24.0) | 38 | (30.6) | 32 | (40.0) | 6 | (13.6) |
| Acute lymphocytic leukemia | 14 | (28.0) | 21 | (16.9) | 14 | (17.5) | 7 | (15.9) |
| Neuroblastoma | 8 | (16.0) | 16 | (12.9) | — | | 16 | (36.4) |
| Myelodysplastic syndrome | — | | 8 | (6.5) | 4 | (5.0) | 4 | (9.1) |
| Non-Hodgkin lymphoma | 1 | (2.0) | 7 | (5.6) | 7 | (8.8) | — | |
| Other | 9 | (18.0) | 15 | (12.1) | 6 | (7.5) | 9 | (20.5) |
| Type of transplant | | | | | | | | |
| Allogeneic | 41 | (80.4) | 102 | (81.6) | 74 | (91.4) | 28 | (63.6) |
| Autologous | 10 | (19.6) | 23 | (18.4) | 7 | (8.6) | 16 | (36.4) |
| GVHD prophylaxis | | | | | | | | |
| None | 13 | (25.5) | 28 | (22.4) | 11 | (13.6) | 17 | (38.6) |
| Tacrolimus | 20 | (39.2) | 71 | (56.8) | 56 | (69.1) | 15 | (34.1) |
| Methotrexate | 8 | (15.7) | 38 | (30.4) | 29 | (35.8) | 9 | (20.5) |
| Cyclosporine | 16 | (31.4) | 19 | (15.2) | 9 | (11.1) | 10 | (22.7) |
| Sirolimus | 5 | (9.8) | 12 | (9.6) | 11 | (13.6) | 1 | (2.3) |
| Other | 20 | (39.2) | 34 | (27.2) | 22 | (27.2) | 12 | (27.3) |

^{a}primary disease was available for 261 patients

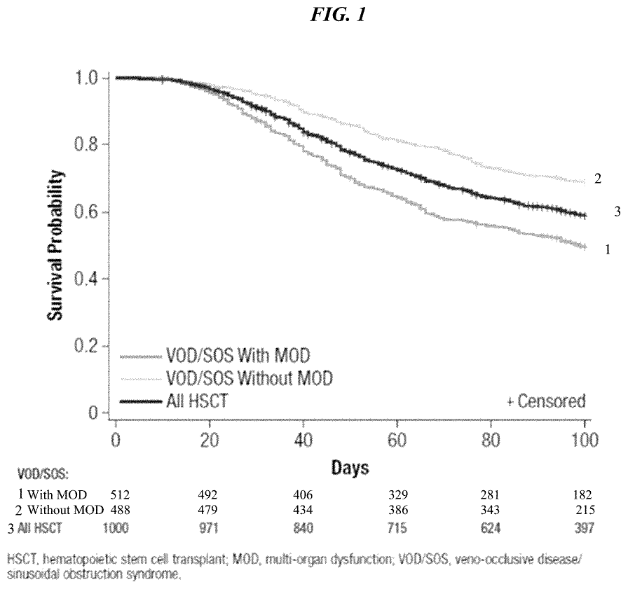

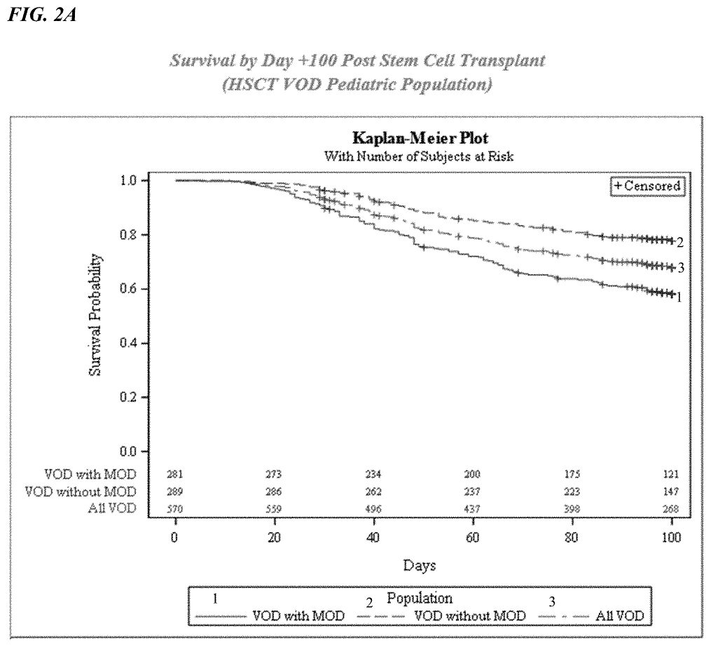

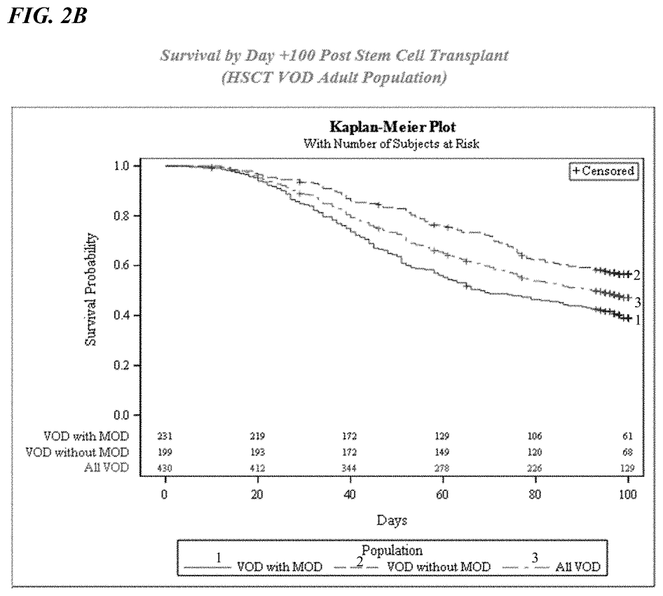

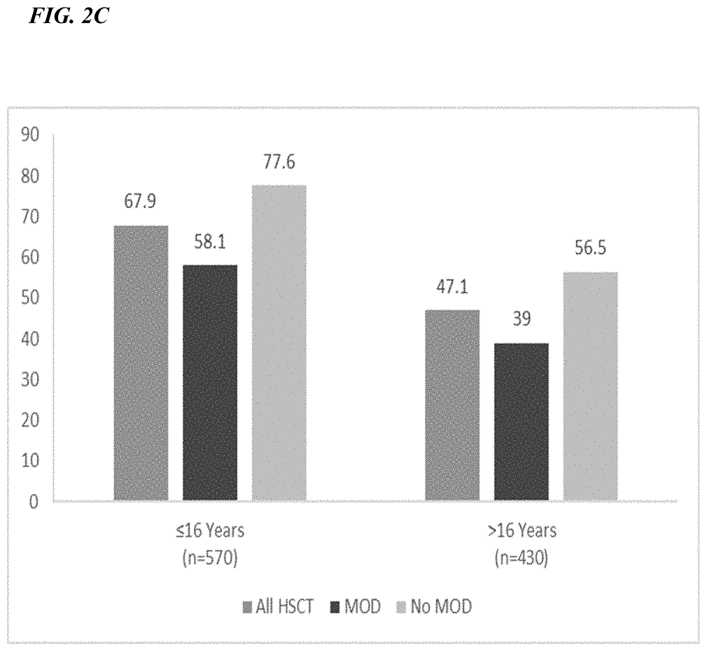

Efficacy. Overall in the HSCT population, Day +100 survival was 58.9% (95% CI, 55.7%-61.9%; FIG. 1). In patients without MOD, Day +100 survival was 68.9% (95% CI, 64.5%-72.9%). In patients with MOD, Day +100 survival was 49.5% (95% CI, 45.0%-53.0%). Estimated survival rates were higher in pediatric patients (67.9%; 95% CI, 63.8-71.6) compared with adults (47.1%; 95% CI, 42.3-51.8) (FIG. 2).

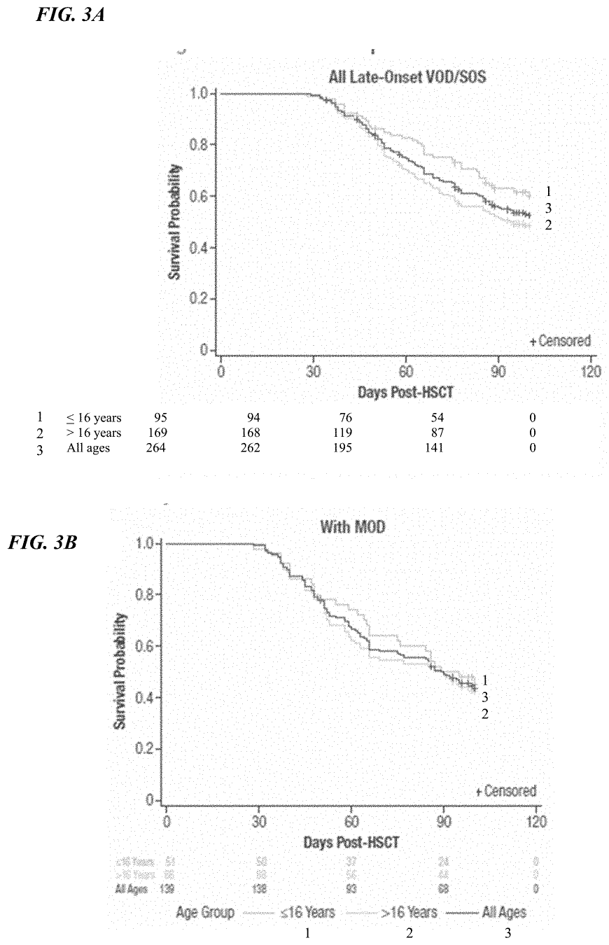

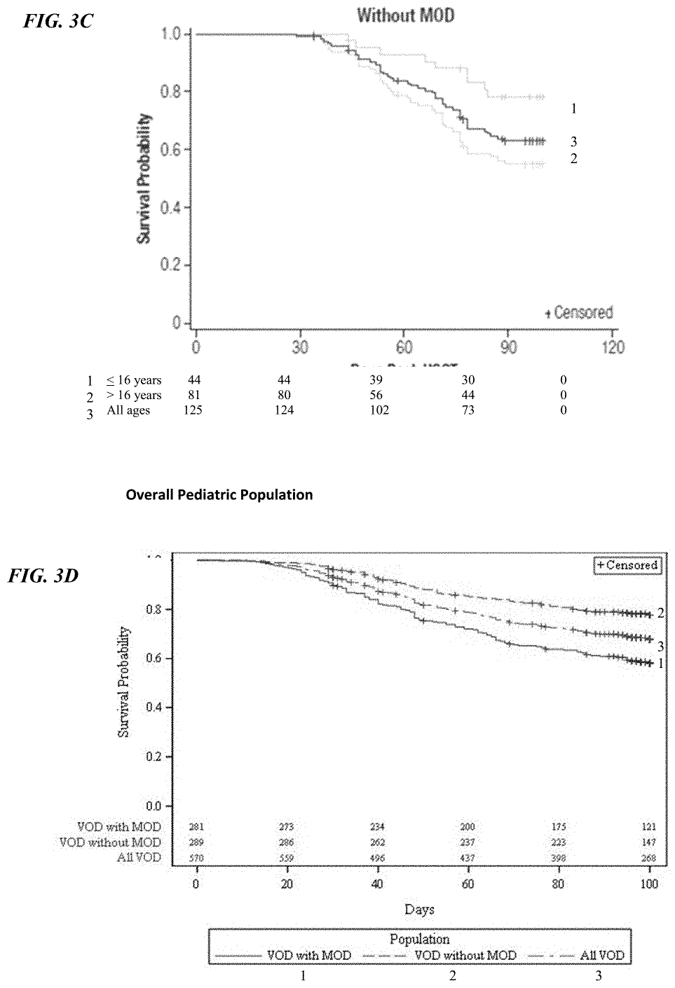

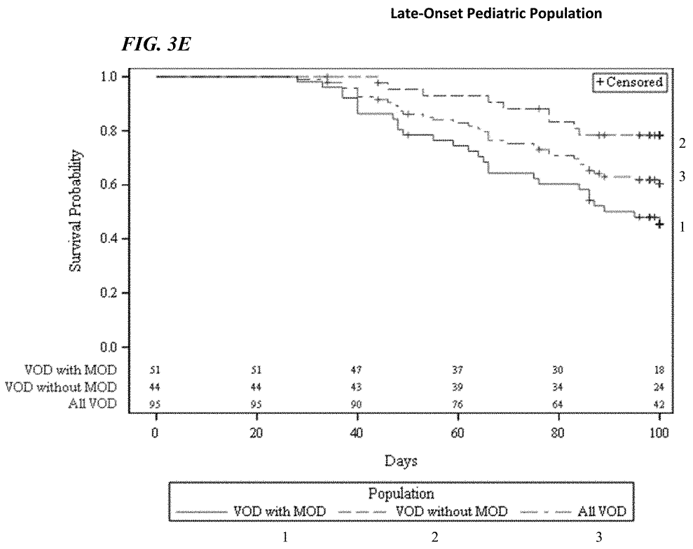

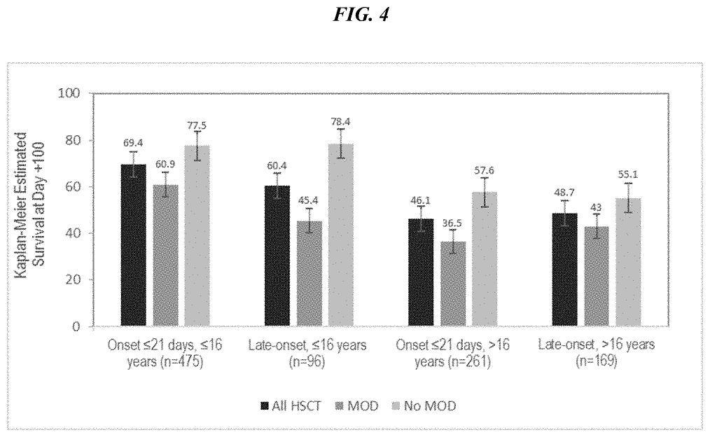

In the total late-onset SOS population, Kaplan-Meier estimated Day +100 survival was 52.8% (95% CI, 46.5%-58.7%) (FIG. 3A). The overall estimated survival was 43.9% (95% CI, 35.4%-52.0%) in the subgroup with MOD, and 63.1% (95% CI, 53.8-70.9) in the subgroup without MOD. For pediatric patients (<16 years) with late-onset SOS, the survival rate was 60.4% (95% CI, 49.5%-69.7%), compared to 67.9% (95% CI, 63.8%-71.6%) for the overall pediatric population (FIGS. 3D and 3E). Survival in late-onset pediatric patients with MOD was 45.4% (95% CI, 31.0%-58.6%), and 78.4% (95% CI, 62.5-88.1) for those without MOD, compared to 58.1% (95% CI, 52.0%-63.7%) and 77.6% (95% CI, 72.2%-82.1%) for the overall pediatric population with MOD and without MOD respectively. Late-onset pediatric patients with MOD had lower day +100 survival rates than pediatric patients diagnosed within 21 days (60.9%, 95% CI, 31.0%-58.6%) (FIG. 4). For adults (>16 years) with late-onset SOS, the survival rate was 48.7% (95% CI, 40.9%-56.0%). Survival in adults with MOD was 43.0% (95% CI, 32.5%-53.0%), and 55.1% (95% CI, 43.5-65.2) for those without MOD.

Safety. In the overall T-IND study of patients having SOS after HSCT, adult patients and patients with MOD generally had higher rates of AEs than pediatric patients and those without MOD (Table 3). In the overall HSCT population, 385 patients (75.2%) had ≥1 AE. 210 patients (21.0%) had ≥1 AE assessed as being at least possibly related to treatment (TRAE). TRAEs lead to discontinuation in 124 (12.4%) patients and death in 28 (2.8%). TRAEs occurring in ≥2% of patients were pulmonary hemorrhage (4.6%), gastrointestinal hemorrhage (3.0%), epistaxis (2.3%), and hypotension (2.0%). In the late-onset cohort, at least 1 adverse event (AE) was reported in 199 (75.4%) patients (Table 4a). Event rates were somewhat more common in patients with MOD and in adults. Other than worsening SOS and MOD, only hypotension (14.0%) occurred in >10% of patients (Table 2); hemorrhagic AEs of all types occurred in 29.5%. Serious AEs were reported in 155 (58.7%) patients. AEs assessed as being at least possibly treatment related (TRAEs) occurred in 55 (20.8%) patients including 33/139 [23.7%] patients with MOD; rates were similar in pediatric (21.1%) and adult patients (20.7%). Most common TRAEs (>3%) were epistaxis (4.2%), pulmonary hemorrhage (4.2%), and gastrointestinal hemorrhage (3.0%). TRAEs leading to study discontinuation occurred in 25 (9.5%) patients; TRAEs leading to death occurred in 10 (3.8%) patients; most commonly pulmonary hemorrhage (1.5%).

TABLE 3
Safety profile of total HSCT population

| | MOD | | | | | | No MOD | | | |
|---|---|---|---|---|---|---|---|---|---|---|
| | Overall | | >16 years | | ≤16 years | | Overall | | >16 years | |
| AE, n (%) | (n = 512) | | (n = 231) | | (n = 281) | | (n = 488) | | (n = 199) | |
| ≥1 AE | 385 | (75.2) | 187 | (81.0) | 198 | (70.5) | 324 | (66.4) | 148 | (74.4) |
| ≥1 Serious AE | 310 | (60.5) | 151 | (65.4) | 159 | (56.6) | 227 | (46.5) | 105 | (52.8) |
| ≥1 Treatment- | 118 | (23.0) | 50 | (21.6) | 68 | (24.2) | 92 | (18.9) | 38 | (19.1) |
| related AE | | | | | | | | | | |
| ≥1 Hemorrhage | 166 | (32.4) | 77 | (33.3) | 89 | (31.7) | 124 | (25.4) | 55 | (27.6) |
| ≥1 Hypotension | 78 | (15.2) | 47 | (20.3) | 31 | (11.0) | 42 | (8.6) | 23 | (11.6) |

| | No MOD | | All HSCT | | | | | |
|---|---|---|---|---|---|---|---|---|
| | ≤16 years | | Overall | | >16 years | | ≤16 years | |
| AE, n (%) | (n = 289) | | (n = 1000) | | (n = 430) | | (n = 570) | |
| ≥1 AE | 176 | (60.9) | 709 | (70.9) | 335 | (77.9) | 374 | (65.6) |
| ≥1 Serious AE | 122 | (42.2) | 537 | (53.7) | 256 | (59.5) | 281 | (49.3) |
| ≥1 Treatment- | 54 | (18.7) | 210 | (21.0) | 88 | (20.5) | 122 | (21.4) |
| related AE | | | | | | | | |
| ≥1 Hemorrhage | 69 | (23.9) | 290 | (29.0) | 132 | (30.7) | 158 | (27.7) |
| ≥1 Hypotension | 19 | (6.6) | 120 | (12.0) | 70 | (16.3) | 50 | (8.8) |

TABLE 4a
Common AEs in late-onset patients (≥5% of patients)

| | All Late-Onset SOS | | | | | |
|---|---|---|---|---|---|---|
| | Overall | | >16 years | | ≤16 years | |
| | (n = 264) | | (n = 169) | | (n = 95) | |
| ≥1 AE | 199 | (75.4) | 132 | (78.1) | 67 | (70.5) |
| Hypotension | 37 | (14.0) | 28 | (16.6) | 9 | (9.5) |
| Multi-organ | 36 | (13.6) | 23 | (13.6) | 13 | (13.7) |
| failure | | | | | | |
| Veno-occlusive | 34 | (12.9) | 25 | (14.8) | 9 | (9.5) |
| liver disease | | | | | | |
| Renal failure | 26 | (9.8) | 20 | (11.8) | 6 | (6.3) |
| Diarrhea | 25 | (9.5) | 21 | (12.4) | 4 | (4.2) |
| Respiratory | 23 | (8.7) | 18 | (10.7) | 5 | (5.3) |
| failure | | | | | | |
| Vomiting | 20 | (7.6) | 17 | (10.1) | 3 | (3.2) |
| Pyrexia | 19 | (7.2) | 12 | (7.1) | 7 | (7.4) |
| Hypoxia | 19 | (7.2) | 11 | (6.5) | 8 | (8.4) |
| Nausea | 17 | (6.4) | 15 | (8.9) | 2 | (2.1) |
| Confusional | 16 | (6.1) | 14 | (8.3) | 2 | (2.1) |
| state | | | | | | |

TABLE 4b
Common AEs in late-onset patients (≥5% of patients)

| SOS with MOD | | | |
|---|---|---|---|
| | Overall | >16 years | ≤16 years |
| | (n = 139) | (n = 88) | (n = 51) |

| ≥1 AE | 112 | (80.6) | 73 | (83.0) | 39 | (76.5) |
|---|---|---|---|---|---|---|
| Hypotension | 25 | (18.0) | 18 | (20.5) | 7 | (13.7) |
| Multi-organ | 23 | (16.5) | 15 | (17.0) | 8 | (15.7) |
| failure | | | | | | |
| Veno-occlusive | 19 | (13.7) | 14 | (15.9) | 5 | (9.8) |
| liver disease | | | | | | |
| Renal failure | 18 | (12.9) | 14 | (15.9) | 4 | (7.8) |
| Diarrhea | 13 | (9.4) | 12 | (13.6) | 1 | (2.0) |
| Respiratory | 11 | (7.9) | 9 | (10.2) | 2 | (3.9) |
| failure | | | | | | |
| Vomiting | 14 | (10.1) | 13 | (14.8) | 1 | (2.0) |
| Pyrexia | 11 | (7.9) | 9 | (10.2) | 2 | (3.9) |
| Hypoxia | 10 | (7.2) | 6 | (6.8) | 4 | (7.8) |
| Nausea | 9 | (6.5) | 9 | (10.2) | — | |
| Confusional | 8 | (5.8) | 7 | (8.0) | 1 | (2.0) |
| state | | | | | | |

TABLE 4c
Common AEs in late-onset patients (≥5% of patients)

| SOS without MOD | | | |
|---|---|---|---|
| | Overall | >16 years | ≤16 years |
| | (n = 125) | (n = 81) | (n = 44) |

| | | | | | | |
|---|---|---|---|---|---|---|
| ≥1 AE | 87 | (69.6) | 59 | (72.8) | 28 | (63.6) |
| Hypotension | 12 | (9.6) | 10 | (12.3) | 2 | (4.5) |
| Multi-organ | 13 | (10.4) | 8 | (9.9) | 5 | (11.4) |
| failure | | | | | | |
| Veno-occlusive | 15 | (12.0) | 11 | (13.6) | 4 | (9.1) |
| liver disease | | | | | | |
| Renal failure | 8 | (6.4) | 6 | (7.4) | 2 | (4.5) |
| Diarrhea | 12 | (9.6) | 9 | (11.1) | 3 | (6.8) |
| Respiratory | 12 | (9.6) | 9 | (11.1) | 3 | (6.8) |
| failure | | | | | | |
| Vomiting | 6 | (4.8) | 4 | (4.9) | 2 | (4.5) |
| Pyrexia | 8 | (6.4) | 3 | (3.7) | 5 | (11.4) |
| Hypoxia | 9 | (7.2) | 5 | (6.2) | 4 | (9.1) |
| Nausea | 8 | (6.4) | 6 | (7.4) | 2 | (4.5) |
| Confusional | 8 | (6.4) | 7 | (8.6) | 1 | (2.3) |
| state | | | | | | |

Conclusions. The T-IND protocol is the largest prospective evaluation of defibrotide treatment for SOS in patients with and without MOD. The final analysis indicates an overall Day +100 survival 58.9%, including a survival rate of 49.5% in those with MOD, a complication typically associated with dismal outcomes. Safety findings were consistent with prior clinical trials. Taken together, these results provide supportive evidence for the clinical utility of defibrotide for treatment of late-onset SOS in pediatric patients with and without MOD.

The overall safety profile for late-onset patients is slightly worse than that the overall T-IND study (Kaplan-Meier estimated Day +100 survival was 52.8% in patients with late onset, compared with 58.9% in the overall post-HSCT cohort). Late-onset and ≤day-21 onset SOS Day +100 estimated survival was similar in the pediatric subgroups and in the adult subgroups. The subgroup having MOD had a Day +100 survival of 43.9% and the subgroup without MOD had a Day +100 survival rate of 63.1. The overall pediatric subgroup showed a significantly higher Day +100 survival rate of 60.4%, with a 45.4% survival rate for the pediatric subgroup having MOD and a 78.4% survival rate for the pediatric subgroup without MOD. Adults, in contrast, had a significantly lower Day +100 survival rate of 48.7, with a 43.0% survival rate for the adult subgroup having MOD and a 55.1% survival rate for the adult subgroup without MOD. Safety profile for these subgroups was similar to the overall T-IND study. Based on the final T-IND data, this post-hoc analysis shows that diagnostic criteria requiring onset by Day +21 would have excluded 26% of all HSCT patients with SOS in this expanded-access program, with more than a third of those being pediatric patients (36.0%). This is likely an underestimate, as the T-IND study did not include enrollment of patients with late-onset SOS in the original protocol. These findings highlight defibrotide's efficacy in treating late-onset patients, especially pediatric patients, and consequently, the importance of including late-onset SOS in diagnostic criteria.

### Example 3

### Determination of Efficacy and Safety for Timing of Defibrotide Initiation

Study Design. This Example utilizes the same T-IND study from Examples 1 and 2, with the primary goal of this Example being the evaluation of the impact of timing of initiation of defibrotide after diagnosis of VOD/SOS following chemotherapy without HSCT. To this end, survival rates were examined by time from VOD/SOS diagnosis to start of defibrotide for two classes: (a) all patients before/after days 1, 2, 3, 4, 7, and 14 post-diagnosis, using Fisher's exact test, and (b) patients starting defibrotide on a particular day—0, 1, 2, 3, 4, 5, 6, 7, 8-14, and ≥15 post-diagnosis—by Cochran-Armitage test for trend across days. The causes of treatment delay were not examined. Safety endpoints included treatment-emergent adverse events (TEAEs) and TRAEs.

Assessments. Day +70 survival (from initiation of defibrotide) was assessed for the cohort of patients who were diagnosed with VOD/SOS and initiated defibrotide treatment by day 30 after the start of primary chemotherapy (n=82). Day +100 survival (from start of chemotherapy) was also assessed for the cohort of patients who were diagnosed with VOD/SOS treatment by day 30 after the start of primary chemotherapy (n=87). Both Day +70 and Day +100 survival was assessed via Kaplan-Meier estimate with 95% confidence interval (CI) calculated using the normal approximation to the binomial distribution. Safety data included new or worsening adverse events (AEs) and symptoms of the original or targeted disease were not to be reported as AEs unless the event was considered serious.

Patients. This Example utilizes the same patients from the T-IND study from Examples 1 and 2, with this Example examining the cohort of patients diagnosed with VOD/SOS and initiated defbrotide treatment by day 30 after the start of primary chemotherapy (not preparatory for HSCT).

Dosing. Defibrotide was administered intravenously 4 times daily (6.25 mg/kg in 2-hour infusions at 6-hour intervals; total 25 mg/kg/day), with a recommended duration of 21 days or more. Doses may have been held to accommodate other procedures (e.g., intermittent hemodialysis or surgery).

### Example 4

### Results and Discussion of Timing of Defibrotide Initiation

Of the 1154 patients treated in the T-IND study, 137 (12.0%) developed SOS post-chemotherapy without undergoing HSCT. 87 of the 137 patients were diagnosed with SOS within 30 days of the start of chemotherapy. 82 of the 137 patients received defibrotide by day 30 after start of chemotherapy. Demographics and disease characteristics of the patients diagnosed with SOS within 30 days of the start of chemotherapy (without HSCT) are presented in Table 5 and patients that received defibrotide by day 30 after start of chemotherapy are presented in Table 6.

TABLE 5
Baseline demographics and disease characteristics of patients diagnosed
with SOS within 30 days of the start of chemotherapy (without HSCT)

| | | | | VOD/SOS | | VOD/SOS | |
|---|---|---|---|---|---|---|---|
| | | All Patients | | without MOD | | with MOD | |
| Variable | | (N = 87) | | (n = 46) | | (n = 41) | |
| Sex, n (%) | | | | | | | |
| Male | | 43 | (49.4) | 22 | (47.8) | 21 | (51.2) |
| Female | | 44 | (50.6) | 24 | (52.2) | 20 | (48.8) |
| Race, n (%) | | | | | | | |
| White | | 59 | (67.8) | 33 | (71.7) | 26 | (63.4) |
| Age at time of | | | | | | | |
| chemotherapy, years | | | | | | | |
| Mean (SD) | | 11.34 | (13.697) | 10.85 | (14.615) | 11.89 | (12.747) |
| Median [range] | | 8 | (0-69) | 6 | (0.3-69) | 8 | (0-67) |
| Adult (>16 years), n (%) | | 18 | (20.7) | 9 | (19.6) | 9 | (22.0) |
| Pediatric (≤16 years), n (%) | | 69 | (79.3) | 37 | (80.4) | 32 | (78.0) |
| 0-23 | months | 10 | (11.5) | 7 | (15.2) | 3 | (7.3) |
| 2-11 | years | 50 | (57.5) | 26 | (56.5) | 24 | (58.5) |
| 12-16 | years | 9 | (10.3) | 4 | (8.7) | 5 | (12.2) |

| | | | | | | |
|---|---|---|---|---|---|---|
| Primary disease (>2%), n (%) | | | | | | |
| ALL | 45 | (51.7) | 29 | (63.0) | 16 | (39.0) |
| AML | 11 | (12.6) | 4 | (8.7) | 7 | (17.7) |
| Neuroblastoma | 5 | (5.7) | 3 | (6.5) | 2 | (4.9) |
| Non-Hodgkin lymphoma | 2 | (2.3) | 1 | (2.2) | 1 | (2.4) |

TABLE 6
Baseline characteristics of patients treated with defibrotide for VOD/SOS
within 30 days of starting non-transplant-associated chemotherapy

| | | | | VOD/SOS | | VOD/SOS | |
|---|---|---|---|---|---|---|---|
| | | All Patients | | without MOD | | with MOD | |
| Variable | | (N = 82) | | (n = 44) | | (n = 38) | |
| Sex, n (%) | | | | | | | |
| Male | | 40 | (48.8) | 21 | (47.7) | 19 | (50.0) |
| Female | | 42 | (51.2) | 23 | (52.3) | 19 | (50.0) |
| Race, n (%) | | | | | | | |
| White | | 55 | (67.1) | 31 | (70.5) | 24 | (63.2) |
| Non-white | | 27 | (32.9) | 13 | (29.5) | 14 | (36.8) |
| Age at time of | | | | | | | |
| chemotherapy,ₐ years | | | | | | | |
| Mean (SD) | | 10.5 | (12.5) | 9.44 | (11.935) | 11.82 | (13.161) |
| Median [range] | | 7.5 | [0.0-68.0] | 5.0 | [0.30-68.0] | 8.00 | [0.0-67.0] |
| Age class, n (%) | | | | | | | |
| Adult (>16 years) | | 16 | (19.5) | 8 | (18.2) | 8 | (21.1) |
| Pediatric (≤16 years) | | 66 | (80.5) | 36 | (81.8) | 30 | (78.9) |
| 0-23 | months | 10 | (15.2) | 7 | (15.9) | 3 | (7.9) |
| 2-11 | years | 49 | (74.2) | 26 | (59.1) | 23 | (60.5) |
| 12-16 | years | 7 | (10.6) | 3 | (6.8) | 4 | (10.5) |

| | | | | | | |
|---|---|---|---|---|---|---|
| Primary disease, n (%)_{b} | | | | | | |
| ALL | 42 | (51.2) | 27 | (61.4) | 15 | (39.5) |
| AML | 11 | (13.4) | 4 | (9.1) | 7 | (18.4) |
| Neuroblastoma | 5 | (6.1) | 3 | (6.8) | 2 | (5.3) |
| Non-Hodgkin lymphoma | 2 | (2.4) | 1 | (2.3) | 1 | (2.6) |
| Other | 22 | (26.8) | 9 | (20.5) | 13 | (34.2) |
| Use of chemotherapeutic | | | | | | |
| agents, n (%) | | | | | | |
| Cyclophosphamide | 44 | (53.7) | 22 | (50.0) | 22 | (57.9) |
| Cytarabine | 42 | (51.2) | 24 | (54.5) | 18 | (47.4) |
| Vincristine | 40 | (47.6) | 23 | (52.3) | 17 | (44.7) |
| Methotrexate | 28 | (34.1) | 16 | (36.4) | 12 | (31.6) |
| Tioguanine | 25 | (30.5) | 17 | (38.6) | 8 | (21.1) |
| Pegaspargase | 24 | (29.3) | 15 | (34.1) | 9 | (23.7) |
| Doxorubicin | 16 | (19.5) | 9 | (20.5) | 7 | (18.4) |
| Etoposide | 15 | (18.3) | 8 | (18.2) | 7 | (18.4) |
| Dexamethasone | 11 | (13.4) | 5 | (11.4) | 6 | (15.8) |
| Combinations of | 11 | (13.4) | 9 | (20.5) | 2 | (5.3) |
| antineoplastic agents | | | | | | |
| Daunorubicin | 9 | (11.0) | 5 | (11.4) | 4 | (10.5) |

ₐIn patients who underwent both nontransplant-related chemotherapy and HSCT (n = 3), chemotherapy was closer to onset of VOD/SOS than HSCT.
_{b}Overall patient population, incidence >2.0%.
_{c}Overall patient population, usage >10.0%.

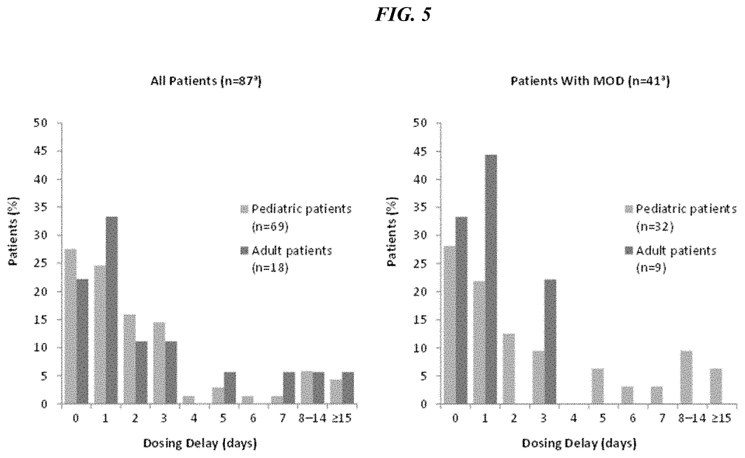

Among the 87 patients diagnosed with SOS within 30 days of the start of chemotherapy, defibrotide was started on the day of diagnosis in 26.4% of patients, and by Day 7 in 89.7% of patients (FIG. 5). Timing of the first defibrotide dose was generally similar in pediatric and adult patients, although treatment initiation in patients with MOD appeared to be somewhat earlier in the small adult population compared with pediatric patients.

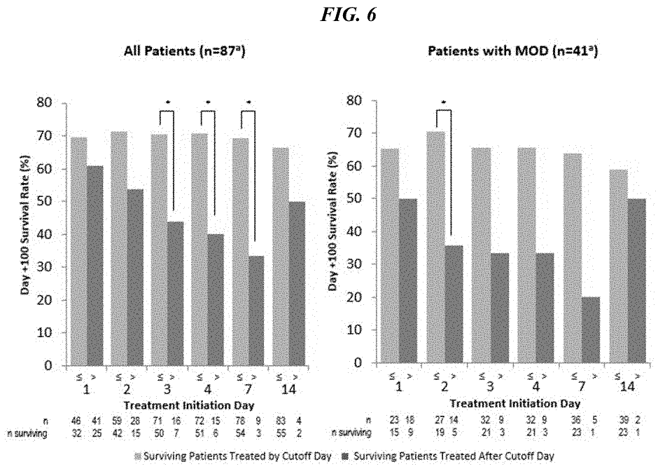

Survival rates at Day +100. In the population-wide analysis of initiation before/after days 1, 2, 3, 4, 7, and 14 post-diagnosis in the overall group of the patients diagnosed with SOS within 30 days of the start of chemotherapy, earlier initiation was associated with higher Day +100 survival rates for all days, a difference that reached statistical significance at days 3, 4, and 7 (P<0.05; FIG. 6), with the survival differences by cutoff date ranging from 10.4% to 37.5%, all favoring earlier treatment. A similar pattern emerged in the subgroup with MOD, which reached significance at day 2.

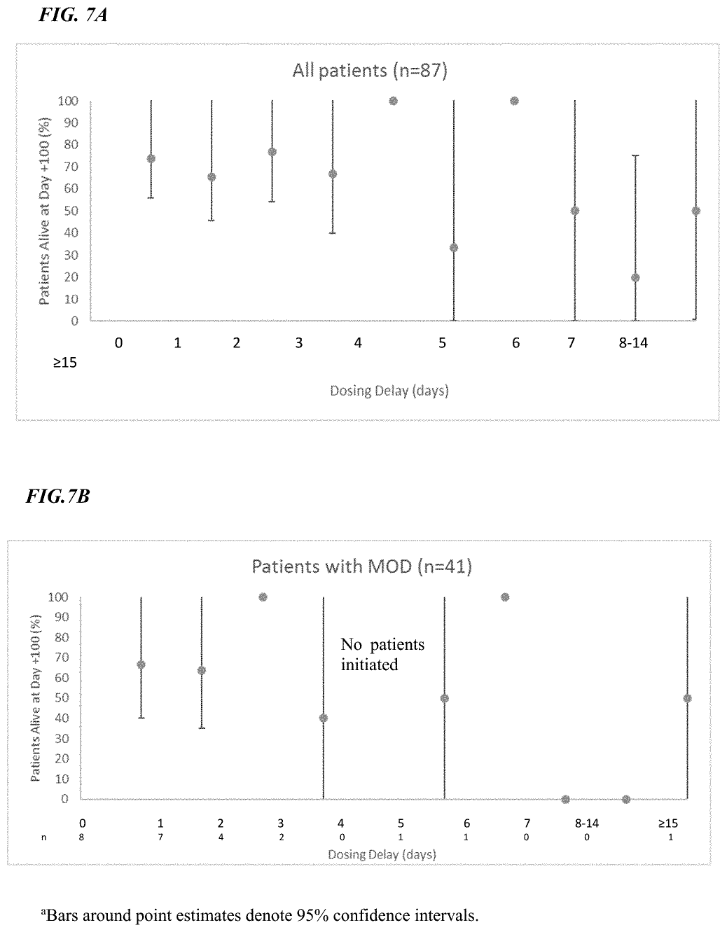

The Cochran-Armitage test was performed to determine a trend for specific initiation days indicated a statistically significant trend over time for higher Day +100 survival with earlier initiation following diagnosis in both the overall group patients diagnosed with SOS within 30 days of the start of chemotherapy (P=0.015) and the MOD subgroup (P=0.041; FIG. 7).

Safety. A total of 59 of the 87 patients (67.8%) in this post-chemotherapy cohort experienced ≥1 treatment-emergent AE (most commonly hypotension or VOD/SOS, n=5 (12.2%) each). Serious AEs occurred in 38 (43.7%) patients and hemorrhagic AEs occurred in 21 (24.1%) patients. 25 (28.7%) patients had ≥1 AE judged at least possibly related to study treatment (TRAE).

In the subgroup of patients diagnosed with VOD/SOS within 30 days of starting primary chemotherapy, earlier defibrotide initiation post-VOD/SOS diagnosis was associated with improved Day +100 survival, confirmed by the Cochran-Armitage test for trend (P=0.015), which was a consistent result found in the 1000 patient HSCT cohort. However, of note, no specific day appears to represent a clinically meaningful cutoff for improvement of Day +100 survival, suggesting that later intervention retains value if treatment must be delayed.

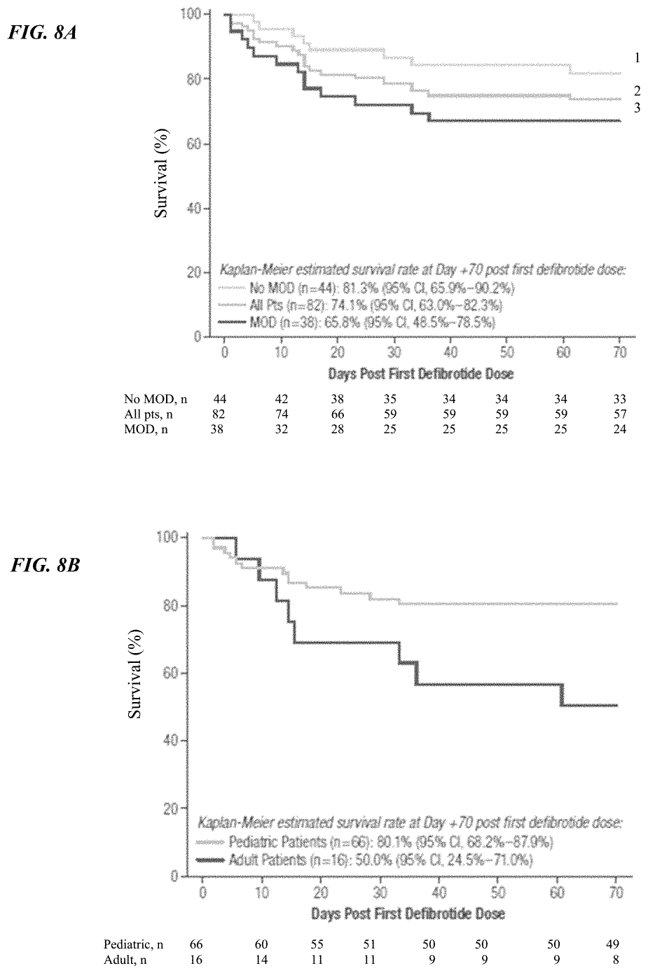

Survival studies of the subgroup of 82 patients that received defibrotide by day 30 after start of chemotherapy was also undertaken. As can be seen from FIGS. 8A and 8B and Table 7, Kaplan-Meier Estimated Survival for patients with and without MOD (FIG. 8A) and pediatric and adult patients (FIG. 8B), show that Kaplan-Meier estimated survival by Day +70 post first defibrotide dose in the total group was 74.1%, 65.8% in the subgroup with MOD, and 81.3% in the subgroup without MOD, and 80.1% in the pediatric subgroup (n=66) and 50.0% in the adult subgroup (n=16). Estimated survival was higher in the 36 pediatric patients without MOD (88.6%) than in the 30 pediatric patients with MOD (70.0%). Day +70 survival was identical for the 8 adult patients with or without MOD (50.0% each).

TABLE 7
Survival at Day +70 after defibrotide initiation among patients treated with defibrotide
for VOD/SOS within 30 days of starting non-transplant-associated chemotherapy.

| | | VOD/SOS | | VOD/SOS |
|---|---|---|---|---|
| | All Patients | without MOD | | with MOD |
| Category | (N = 82) | (n = 44) | | (n = 38) |
| Alive, n (%) | 58 (70.7) | 33 | (75.0) | 25 (65.8) |
| Dead, n (%) | 21 (25.6) | 8 | (18.2) | 13 (34.2) |

| | | | |
|---|---|---|---|
| Kaplan-Meierₐ | 74.1 | 81.3 | 65.8 |
| Survival, % | 63.0-82.3 | 65.9-90.2 | 48.5-78.5 |
| 95% CI | | | |

Age-

stratified

| Survival | Age ≤16 | | Age >16 | Age ≤16 | | Age >16 | Age ≤16 | | Age >16 |
|---|---|---|---|---|---|---|---|---|---|
| Category | (n = 66) | | (n = 16) | (n = 36) | | (n = 8) | (n = 30) | | (n = 8) |
| Alive, n (%) | 50 | (75.8) | 8 (50.0) | 29 | (80.6) | 4 (50.0) | 21 | (70.0) | 4 (50.0) |
| Dead, n (%) | 13 | (19.7) | 8 (50.0) | 4 | (11.1) | 4 (50.0) | 9 | (30.0) | 4 (50.0) |

| | | | | | | | | |
|---|---|---|---|---|---|---|---|---|
| Unknown, n (%) | 3 | (4.5%) | — | 3 | (8.3) | — | — | — |

| | | | | | | |
|---|---|---|---|---|---|---|
| Kaplan-Meierₐ | 80.1 | 50.0 | 88.6 | 50.0 | 70.0 | 50.0 |
| Survival, % | 68.2-87.9 | 24.5-71.0 | 72.4-95.6 | 15.2-77.5 | 50.3-83.1 | 15.2-77.5 |
| 95% CI | | | | | | |

ₐEstimated survival at Day +70 after defibrotide initiation

The safety profile for this cohort was also examined, and AEs were reported in 54/82 patients (65.9%) as shown in Table 8. AEs assessed as being at least possibly related to defibrotide occurred in 22 patients (26.8%), as shown in Table 9. The incidence of AEs and of serious AEs was notably higher among patients with MOD (84.2% and 57.9%, respectively) than among patients without MOD (50.0% and 25.0%, respectively). Six patients (7.3%) withdrew from the study due to an AE considered by the investigator to be at least possibly related to defibrotide; except for 1 case each of hypotension and intraventricular hemorrhage, these AEs were primarily gastrointestinal disorders (3.7%; gastric, gastrointestinal and mouth hemorrhage, each in 1 patient with MOD [2.6%]), followed by respiratory, thoracic, and mediastinal disorders (2.4%; epistaxis and pulmonary hemorrhage, each in 1 patient with MOD [2.6%]). Across all patients, 1 death (1.2%) from pulmonary hemorrhage and hypotension in a patient with MOD, was considered to be related to defibrotide.

AEs by System Organ Class and Preferred Term consistent with hemorrhage were reported in 18 of 82 patients (22.0%). The events in more than 1 patient were pulmonary hemorrhage (in 5 patients; 6.1%), epistaxis and mouth hemorrhage (each in 3 patients; 3.7%), and hematochezia (in 2 patients; 2.4%). The organ systems most commonly affected in these events were the gastrointestinal and respiratory systems (each in 9 patients; 11.0%). Hemorrhagic AEs were more common among patients with MOD (34.2%) than among patients without MOD (11.4%).

Overall, the safety profile was consistent with that previously reported, and in the overall population of the T-IND program (N=1154).

TABLE 8
Adverse Events in patients that received defibrotide
by day 30 after start of chemotherapy.

| | All Patients in | | | | | |
|---|---|---|---|---|---|---|
| | CHEMO-30 | | Patients | | Patients | |
| | Analysis Set | | With MOD | | Without MOD | |
| Adverse Event, n (%) | (N = 82) | | (n = 38) | | (n = 44) | |
| ≥1 AE | 54 | (65.9) | 32 | (84.2) | 22 | (50.0) |
| Multi-organ | 7 | (8.5) | 4 | (10.5) | 3 | (6.8) |
| dysfunction^{a} | | | | | | |
| Hypotension^{a} | 7 | (8.5) | 5 | (13.2) | 2 | (4.5) |
| Vomiting^{a} | 6 | (7.3) | 4 | (10.5) | 2 | (4.5) |
| Respiratory failure^{a} | 6 | (7.3) | 2 | (5.3) | 4 | (9.1) |
| Veno-occlusive | 5 | (6.1) | 5 | (13.2) | 0 | |
| disease^{a} | | | | | | |
| Febrile neutropenia^{a} | 5 | (6.1) | 3 | (7.9) | 2 | (4.5) |
| Hypokalemia^{a} | 5 | (6.1) | 3 | (7.9) | 2 | (4.5) |
| Hypoxia^{a} | 5 | (6.1) | 4 | (10.5) | 1 | (2.3) |
| Pulmonary hemorrhage^{a} | 5 | (6.1) | 5 | (13.2) | 0 | |
| ≥1 Serious AE | 33 | (40.2) | 22 | (57.9) | 11 | (25.0) |
| Multi-organ failure^{b} | 7 | (8.5) | 4 | (10.5) | 3 | (6.8) |
| Veno-occlusive | 5 | (6.1) | 5 | (13.2) | 0 | |
| disease^{b} | | | | | | |
| Hypoxia^{b} | 5 | (6.1) | 4 | (10.5) | 1 | (2.3) |
| Respiratory failure^{b} | 5 | (6.1) | 2 | (5.3) | 3 | (6.8) |
| Pulmonary hemorrhage^{b} | 4 | (4.9) | 4 | (10.5) | 0 | |
| Febrile neutropenia^{b} | 3 | (3.7) | 3 | (7.9) | 0 | |
| Encephalopathy^{b} | 3 | (3.7) | 1 | (2.6) | 2 | (4.5) |
| Renal failure^{b} | 3 | (3.7) | 2 | (5.3) | 1 | (2.3) |
| Hypotension^{b} | 3 | (3.7) | 3 | (7.9) | 0 | |
| Hepatic failure^{b} | 2 | (2.4) | 2 | (5.3) | 0 | |
| Sepsis^{b} | 2 | (2.4) | 2 | (5.3) | 0 | |
| ≥1 Hemorrhagic AE | 18 | (22.0) | 13 | (34.2) | 5 | (11.4) |
| Pulmonary hemorrhage^{b} | 5 | (6.1) | 5 | (13.2) | 0 | |
| Epistaxis^{b} | 3 | (3.7) | 3 | (7.9) | 0 | |
| Mouth^{b} | 3 | (3.7) | 2 | (5.3) | 1 | (2.3) |
| Hematochezia^{b} | 2 | (2.4) | 0 | | 2 | (4.5) |
| ≥1 AE leading to | 13 | (15.9) | 10 | (26.3) | 3 | (6.8) |
| discontinuation | | | | | | |
| ≥1 AE leading to | 20 | (24.4) | 12 | (31.6) | 8 | (18.2) |
| discontinuation | | | | | | |

^{a}>5% of patients;
^{b}≥2% of patients.

TABLE 9
Common Treatment-Related Adverse Events (≥2%
of patients) in patients that received defibrotide
by day 30 after start of chemotherapy.

| | All Patients in | | | | | |
|---|---|---|---|---|---|---|
| | CHEMO-30 | | Patients | | Patients | |
| | Analysis Set | | With MOD | | Without MOD | |
| Adverse Event, n (%) | (N = 82) | | (n = 38) | | (n = 44) | |
| ≥1 Treatment-related AE | 22 | (26.8) | 13 | (34.2) | 9 | (20.5) |
| Pulmonary hemorrhage | 3 | (3.7) | 3 | (7.9) | 0 | |
| Mouth hemorrhage | 3 | (3.7) | 2 | (5.3) | 1 | (2.3) |

| | | | | | |
|---|---|---|---|---|---|
| Hematochezia | 2 | (2.4) | 0 | 2 | (4.5) |
| Nausea | 2 | (2.4) | 0 | 2 | (4.5) |
| Encephalopathy | 2 | (2.4) | 0 | 2 | (4.5) |

| | | | | | |
|---|---|---|---|---|---|
| Epistaxis | 2 | (2.4) | 2 | (5.3) | 0 |
| Hypotension | 2 | (2.4) | 2 | (5.3) | 0 |
| Any defibrotide-related_{b} | 6 | (7.3) | 6 | (15.8) | — |
| AE leading to | | | | | |
| discontinuation | | | | | |
| Gastric hemorrhage | 1 | (1.2) | 1 | (2.6) | — |
| Gastrointestinal | 1 | (1.2) | 1 | (2.6) | — |
| hemorrhage | | | | | |
| Mouth hemorrhage | 1 | (1.2) | 1 | (2.6) | — |
| Intraventricular | 1 | (1.2) | 1 | (2.6) | — |
| hemorrhage | | | | | |
| Epistaxis | 1 | (1.2) | 1 | (2.6) | — |
| Pulmonary hemorrhage | 1 | (1.2) | 1 | (2.6) | — |
| Hypotension | 1 | (1.2) | 1 | (2.6) | — |
| Any defibrotide-related_{b} | 1 | (1.2) | 1 | (2.6) | — |
| AE leading to death | | | | | |
| Pulmonary hemorrhage | 1 | (1.2) | 1 | (2.6) | — |
| Hypotension | 1 | (1.2) | 1 | (2.6) | — |

ₐThe listed events were each reported in >1 patient.
_{b}At least possibly related, in the judgment of the investigator.

### Example 5

### A Phase 3, Randomized, Adaptive Study Comparing the Efficacy and Safety of Defibrotide v Best Supportive Care (BSC) in the Prevention of SOS in Adult and Pediatric Patients Undergoing Hematopoietic Stem Cell Transplant

This study will compare the efficacy and safety of defibrotide and best supportive care (BSC) in the prevention of SOS in adult and pediatric patients undergoing HSCT who are at high risk or very high risk of developing SOS, as diagnosed using the modified Seattle criteria.

The primary efficacy endpoint is SOS-free survival by Day +30 post-HSCT in patients who are high risk, or very high risk, for developing SOS. The key secondary objective of the study is to compare the efficacy of defibrotide prophylaxis in addition to BSC (DP arm) v BSC alone (BSC arm) for the prevention of SOs as measured by SOS-free survival by Day +100 post-HSCT in patients who are high risk or very high risk for developing SOS. Other secondary objectives include: 1) to compare the efficacy of defibrotide prophylaxis in addition to BSC (DP arm) v BSC alone (BSC arm) on additional variables: a) incidence of SOS by Day +30 post-HSCT; b) SOS-free survival by Day +180 post-HSCT; c) non-relapse mortality by Day +100 and by Day +180 post-HSCT; d) incidence of SOS-associated multi-organ dysfunction (i.e. severe SOS) by Day +30 and by Day +100 post-HSCT (in those patients who develop SOS); e) proportion of patients who have resolution of SOS by Day +180 post-HSCT and time to resolution of SOS (in those patients who develop SOS); 0 incidence of SOS after Day +30 post-HSCT, by Day +100, and by Day +180 post-HSCT; 2) to compare the health-related quality of life using the following questionnaires: 5-level EuroQol-5D (adults only); EuroQol-5D for Youth, proxy version 1 (pediatric patients 4 to 7 years old); EQ-SD-Y, self-report version (pediatric patients 8 to ≤16 years old); 3) to characterize the pharmacodynamics of defibrotide; 4) to compare the overall safety of defibrotide in addition to BSC v BSC alone, including adverse event (AE) profile, serious adverse even (SAE) profile, laboratory abnormalities, and vital signs (including peri-infusional vital signs for patients who receive defibrotide); 5) to compare the overall safety of defibrotide in addition to BSC v BSC alone by comparing the incidence of grades 2, 3, and 4 acute graft-versus-host-disease (GvHD) by Day +30, Day +100, and Day +180 post-HSCT, and the incidence of chronic GvHD at Day +180 post-HSCT; and 6) to compare graft failure and time to neutrophil and platelet engraftment.

A total of 400 patients are planned for enrollment to ensure completion of approximately 360 patients. Screening procedures will be performed within 14 days of the scheduled start of the patients HSCT conditioning regimen. Eligible patients will be randomly assigned to receive defibrotide prophylaxis 25 mg/kg/day in addition to BSC (“DP arm”) or BSC alone (“BSC arm”) in a 1:1 ratio. Randomization will be stratified according to risk of developing SOS (high risk or very high risk), age (>16 years or ≤16 years), and country using an interactive web response system (IWRS). Enrollment of those patients meeting high-risk criteria will be capped at 65% of the total enrollment using IWRS.

All patients enrolled in the study will receive individualized standard of care therapy based on local institutional guidelines and patient need (e.g. Best Supportive Care or BSC). Patients randomized to the defibrotide prophylaxis arm (DP) will also receive defibrotide prophylaxis. Administration of defibrotide to patients in the DO arm will begin the day before the first day of the conditioning regimen (i.e. Study Day 1) and will continue (for those patients with a SOS diagnosis) for at least 21 days and end no later than Day +30 post-HSCT. Twenty-one days of defibrotide prophylaxis corresponds to approximately Day +15 post-HSCT, assuming 5 days of conditioning therapy before HSCT. For patients in the BSC arm, administration of BSC will begin on the first day of conditioning (i.e. Study Day 1) and continue until Day +30 post-HSCT or hospital discharge, whichever is sooner, or diagnosis of SOS if applicable. Patients receiving BSC should not receive defibrotide as part of their BSC regimen.

Defibrotide solution will be administered intravenously at a dose of 25 mg/kg/day, divided into four doses of 6.25 mg/kg/dose given as 2-hour infusions every six hours. Each of the 4 divided doses per day will be rounded to the nearest 10 mg for adults (>35 kg) and the nearest 5 mg for children (≤35 kg).

Patients randomized to receive defibrotide prophylaxis, defibrotide will be administered starting on the day before the first day of the conditioning regimen (i.e. Study Day 1) for a recommended minimum of 21 days and ending no later than Day +3-post-HSCT. Patients in this arm will also receive individualized standard of care therapy based on local institutional guidelines and patient needs. Patients must undergo Day +15 post-HSCT assessments before hospital discharge. If the patient leaves the hospital before Day +15 post-HSCT, the assessment must be completed on the day of study/study drug discontinuation.

Best supportive care for patients in the BSC arm will be administered on the first day of conditioning (i.e. Study Day 1) and will continue until Day +30-post-HSCT or hospital discharge, whichever is sooner, or diagnosis of SOS if applicable.

For patients in either the DP or BSC arm who develop SOS per modified Seattle criteria, defibrotide will be administered until resolution of SOS or hospital discharge, whichever is sooner, and may continue beyond Day +30 post-HSCT.

If patients in either the DP or BSC arm develop SOS per the modified Seattle criteria, they may receive rescue defibrotide treatment for SOS as prespecified in the informed consent and/or assent forms. For patients in either arm who develop SOS, defibrotide for treatment of SOS should be administered until resolution of SOS or hospital discharge.

Patients will continue to be monitored for development of late-onset SOS through Day +180 post-HSCT. Patients who develop clinical signs and symptoms of VOD after hospital discharge/Day +30 post-HSCT will require more frequent monitoring and re-admission to the hospital at the investigator's discretion.

Efficacy will be assessed through monitoring of hepatic function for development of SOS, relapse of disease, SOS-associated MOD (i.e. severe SOS), resolution of SOS and survival. Other assessments include health-related quality of life, hospital resource utilization, and measurement of biomarkers in blood indicative of potential predictive or diagnostic SOS and/or GVHD biomarkers, and antidrug binding and neutralizing antibodies.

The study will last approximately 5 years, with an estimated enrollment period of 4.5 years for the maximum of 600 patients and duration of participation for each patient lasting approximately 6 months.

### INCORPORATION BY REFERENCE

All references, applications, publications, patents, and patent publications cited are incorporated by reference herein in their entirety for all purposes.

### REFERENCES

Kantarjian H M et al. N Engl J Med. 2016; 375(8):740-753.
Coppell J A, et al. Biol Blood Marrow Transplant. 2010; 16(2):157-168
Wadleigh M et al. Blood. 2003; 102(5):1578-1582; Kantarjian H M et al. N Engl J Med. 2016; 375(8):740-753; Satti M B et al. J Clin Pathol. 1982; 35(10):1086-1091.
5. Choi A et al. Cancer Res Treat. 2016; 48(8):1443-1447
Mohty M, et al. Bone Marrow Transplant. 2015; 50(6):781-789.
Defitelio (defibrotide). Summary of Product Characteristics. Available at: http://www.ema.europa.eu/docs/en_GB/document_library/EPAR_-_Product_Information/human/
002393/WC500153150.pdf. Accessed May 9, 2017; DefitelioR (defibrotide sodium) Prescribing Information. Jazz Pharmaceuticals, Inc. Palo Alto, Calif. 94304. March 2016
Mohty M, et al. Bone Marrow Transplant. 2015; 50(6):781-789
Jones R J, Lee K S, Beschorner W E, et al. Venoocclusive disease of the liver following bone marrow transplantation. Transplantation. 1987; 44(6):778-783; McDonald G B, Hinds M S, Fisher L D, et al. Veno-occlusive disease of the liver and multiorgan failure after bone marrow transplantation: a cohort study of 355 patients. Ann Intern Med. 1993; 118(4):255-267.
Corbacioglu S, et al. Lancet. 2012; 379(9823):1301-1309.
O'Donnell P H, et al. Leuk Lymphoma. 2010; 51(12):2240-2249; Lee J L, et al. Biol Blood Marrow Transplant. 1999; 5(5):306-315; Hasegawa S, et al. Bone Marrow Transplant. 1998; 22, 1191-1197
Hasegawa S, et al. Bone Marrow Transplant. 1998; 22, 1191-1197; Pai R K, et al. Leuk Lymphoma. 2012; 53(8):1552-1557
Carreras E, et al. Biol Blood Marrow Transplant. 2007; 13(12):1448-1454
Cheuk et al. Interventions for prophylaxis of hepatic veno-occlusive disease in people undergoing haemotopoietic stem cell transplantation. The Cochrane database of systematic reviews. May 27 2015(5): CD009311.
Imran H, Tleyjeh I M, Zirakzadeh A, et al. Use of prophylactic anticoagulation and the risk of hepatic veno-occlusive disease in patients undergoing hematopoietic stem cell transplantation: a systematic review and meta-analysis. Bone marrow transplantation. April 2006; 37(7): 677-686.

Mohty M, Malard F, Abecassis M, et al. Sinusoidal obstruction syndrome/veno-occlusive disease: current situation and perspectives-a position statement from the European Society for Blood and Marrow Transplantation (EBMT). Bone marrow transplantation. June 2015; 50(6): 781-789.

Attal M, Huguet F, Rubie H, et al. Prevention of hepatic veno-occlusive disease after bone marrow transplantation by continuous infusion of low-dose heparin: a prospective, randomized trial. Blood. Jun. 1, 1992; 79(11):2834-2840.
Or, O., Nagler, A., Shpilberg, O., Elad, S., Naparstek, E., Kapelushnik, J., Cass, Y., Gillis, S., Chetrit, A., Slavin, S. & Eldor, A. (1996) Low molecular weight heparin for the prevention of veno-occlusive disease of the liver in bone marrow transplantation patients. Transplantation, 61, 1067-1071
Marsa-Vila L, Gorin N C, Laporte J P, et al. Prophylactic heparin does not prevent liver veno-occlusive disease following autologous bone marrow transplantation. European journal of haematology. November 1991; 47(5):346-354.
Dignan F L, Wynn R F, Hadzic N, et al. BCSH/BSBMT guideline: diagnosis and management of veno-occlusive disease (sinusoidal obstruction syndrome) following haematopoietic stem cell transplantation. Br J Haematol. November 2013; 163(4):444-457.
Ruutu T, Eriksson B, Remes K, et al. Ursodeoxycholic acid for the prevention of hepatic complications in allogeneic stem cell transplantation. Blood. Sep. 15, 2002; 100(6):1977-1983.
Tay J, Tinmouth A, Fergusson D, et al. Systematic review of controlled clinical trials on the use of ursodeoxycholic acid for the prevention of hepatic veno-occlusive disease in hematopoietic stem cell transplantation. Biol Blood Marrow Transplant. February 2007; 13(2):206-217.
Tsochatzis E A, Feudjo M, Rigamonti C, et al. Ursodeoxycholic acid improves bilirubin but not albumin in primary biliary cirrhosis: further evidence for nonefficacy. BioMed research international. 2013; 2013:139763.
Corbacioglu et al. Defibrotide for the treatment of hepatic veno-occlusive disease in children after hematopoietic stem cell transplantation. Expert Rev Hematol. June 2012; 5(3):291-302.
Corbacioglu et al. 2006. Stem cell transplantation in children with infantile osteopetrosis is associated with a high incidence of VOD, which could be prevented with defibrotide. Bone marrow transplantation. October 2006; 38(8); 547-553.
Richardson P G, Smith A R, Triplett B M, et al. Earlier defibrotide initiation post-diagnosis of veno-occlusive disease/sinusoidal obstruction syndrome improves Day +100 survival following hematopoietic stem cell transplantation. British Journal of Haematology. 2017a.
Chalandon Y, Roosnek E, Mermillod B, et al. Prevention of veno-occlusive disease with defibrotide after allogeneic stem cell transplantation. Biol Blood Marrow Transplant. May 2004; 10(5):347-354.
Dignan F, Gujral D, Ethell M, et al. Prophylactic defibrotide in allogeneic stem cell transplantation: minimal morbidity and zero mortality from veno-occlusive disease. Bone marrow transplantation. July 2007; 40(1):79-82.

Qureshi A, Marshall L, Lancaster D. Defibrotide in the prevention and treatment of veno-occlusive disease in autologous and allogeneic stem cell transplantation in children. Pediatr Blood Cancer. April 2008; 50(4):831-832.

1. A method for treating a patient diagnosed as having sinusoidal obstruction syndrome, comprising administering a therapeutically effective amount of defibrotide to the patient,
wherein the patient is from about 0 years of age to about 16 years of age, and
wherein the patient was diagnosed as having sinusoidal obstruction syndrome at least 21 days after the patient has undergone hematopoietic stem cell transplantation, and
wherein one or more administrations of the defibrotide are given to the patient over a treatment period.

2. The method of claim 1, wherein the patient was not diagnosed as having sinusoidal obstruction syndrome within 21 days after the patient has undergone hematopoietic stem cell transplantation.

3. The method of claim 1, wherein the patient has multiple organ dysfunction.

4. (canceled)

5. The method of claim 1, wherein the one or more administrations of the defibrotide begins on or after 22 days after the patient has undergone hematopoietic stem cell transplantation.

6. The method of claim 1, wherein the one or more administrations of the defibrotide begins on or after 30 days after the patient has undergone hematopoietic stem cell transplantation.

7. The method of claim 1, wherein the treatment period lasts at least 21 days.

8. The method of claim 1, wherein the defibrotide is administered in an amount about 25 mg per kilogram of body weight per day.

9. The method of claim 1, wherein the defibrotide is administered in four doses per day.

10. The method of claim 1, wherein the defibrotide is administered intravenously.

11. The method of claim 1, wherein the patient is administered defibrotide intravenously in an amount of about 6.25 mg per kilogram of body weight about every 6 hours.

12. The method of claim 1, wherein the patient is selected from a group consisting of about 0 months to about 23 months of age, about 2 years to about 11 years of age, and about 12 years to about 16 years of age.

13.-14. (canceled)

15. A method for treating a patient diagnosed as having sinusoidal obstruction syndrome, comprising administering a therapeutically effective amount of defibrotide to the patient,
wherein the patient has undergone at least one chemotherapy treatment and has not undergone hematopoietic stem cell transplantation, and
wherein one or more administrations of the defibrotide are given to the patient over a treatment period.

16. The method of claim 15, wherein the one or more administrations of the defibrotide begins on 0, 1, 2, 3, 4, 5, 6, or 7 days after that the patient diagnosed as having sinusoidal obstruction syndrome.

17. The method of claim 15, wherein the treatment period lasts at least 21 days.

18. The method of claim 15, wherein the patient is an adult or a pediatric patient.

19. The method of claim 15, wherein the patient has multiple organ dysfunction.

20. The method of claim 15, wherein the defibrotide is administered in an amount about 25 mg per kilogram of body weight per day.

21. The method of claim 15, wherein the defibrotide is administered in four doses per day.

22. The method of claim 15, wherein the defibrotide is administered intravenously.

23. The method of claim 15, wherein the patient is administered defibrotide intravenously in an amount of about 6.25 mg per kilogram of body weight every 6 hours.
